(19) United States
(12) Patent Application Publication
(10) Pub. No.: US 2011/0028398 A1
(43) Pub. Date: Feb. 3, 2011

(54) METHODS FOR TREATING AND LIMITING FIBROTIC DISORDERS AND KELOIDS

(75) Inventors: Luciana Biagini Lopes, Clifton Park, NY (US); Elizabeth J. Furnish, Tempe, AZ (US); Charles Robert Flynn, Chandler, AZ (US); Padmini Komalavilas, Tempe, AZ (US); Alyssa Panitch, W. Lafayette, IN (US); Colleen M. Brophy, Nashville, TN (US)

Correspondence Address: FOLEY & LARDNER LLP, 111 HUNTINGTON AVENUE, 26TH FLOOR, BOSTON, MA, 02199-7610, US

(73) Assignee: The Arizona Board of Regents, a body corporate acting for and on behalf of Arizona State Universi, Scottsdale, AZ (US)

(21) Appl. No.: 12/305,885

(22) PCT Filed: Jul. 10, 2007
(86) PCT No.: PCT/US07/73144

§ 371 Date: Oct. 21, 2010

Related U.S. Application Data
(60) Provisional application No. 60/830,279, filed on Jul. 12, 2006.
(60) Provisional application No. 60/849,041, filed on Oct. 2, 2006.

(51) Int. Cl. A61K 38/16; A61P 9/04; A61P 17/02; A61P 27/02; A61K 38/10; A61P 13/12; A61P 1/16; A61P 19/04

(52) U.S. Cl. 514/15.4; 514/16.4; 514/18.6; 514/20.8; 514/21.5; 514/21.4

(57) ABSTRACT

The present invention provides methods for treating and/or limiting fibrotic disorders and/or treating or limiting scars selected from the group consisting of keloids and hypertrophic scars comprising administering to an individual in need thereof an amount effective to treat and/or limit scars selected from the group consisting of keloids and hypertrophic scars of a polypeptide comprising an HSP20-related polypeptide.

### RELATED APPLICATIONS

This application claims priority to U.S. provisional patent application Ser. Nos. 60/830,279 filed Jul. 12, 2006 and 60/849,041 filed Oct. 2, 2006, both of which are incorporated by reference herein in their entirety.

### STATEMENT OF GOVERNMENT SUPPORT

This work was supported at least in part by NIH grant K25HL074968 and NIH STTR grant 6 R42 HL071309-03. The U.S. government has certain rights in the invention.

### BACKGROUND

Keloids and hypertrophic scars are fibroproliferative abnormal healing disorders characterized by excessive scarring due to excessive production, deposition and contraction of extracellular matrix, which results in functional and cosmetic deformity (Leask and Abraham, 2004). There is no current effective treatment for these conditions.

### SUMMARY OF THE INVENTION

In a first aspect, the present invention provides methods for treating and/or limiting fibrotic disorders comprising administering to an individual in need thereof an amount effective to treat and/or limit fibrotic disorders of a polypeptide comprising a sequence according to general formula I:

X1-A(X2)APLP-X3 (SEQ ID NO: 302 and SEQ ID NO: 316)

wherein X1 is 0-14 amino acids of the sequence of heat shock protein 20 between residues 1 and 14 of SEQ ID NO: 298;

X2 is selected from the group consisting of S, T, Y, D, E, hydroxylysine, hydroxyproline, phosphoserine analogs, and phosphotyrosine analogs; and

X3 is selected from the group consisting of (a) 0-140 amino acids of residues 21 and 160 of SEQ ID NO:298; and (b) 0, 1, 2, or 3 amino acids of a sequence of genus Z1-Z2-Z3, wherein Z1 is selected from the group consisting of G and D;

Z2 is selected from the group consisting of L and K; and

Z3 is selected from the group consisting of S, T, and K.

In a second aspect, the present invention provides methods for treating and/or limiting scars selected from the group consisting of keloids and hypertrophic scars comprising administering to an individual in need thereof an amount effective to treat and/or limit scars selected from the group consisting of keloids and hypertrophic scars of a polypeptide comprising a sequence according to general formula I:

X1-A(X2)APLP-X3 (SEQ ID NO: 302 and SEQ ID NO: 316)

wherein X1 is 0-14 amino acids of the sequence of heat shock protein 20 between residues 1 and 14 of SEQ ID NO: 298;

X2 is selected from the group consisting of S, T, Y, D, E, hydroxylysine, hydroxyproline, phosphoserine analogs, and phosphotyrosine analogs; and

X3 is selected from the group consisting of (a) 0-140 amino acids of residues 21 and 160 of SEQ ID NO:298; and (b) 0, 1, 2, or 3 amino acids of a sequence of genus Z1-Z2-Z3, wherein Z1 is selected from the group consisting of G and D;

In various embodiments of the first and/or second aspect of the invention, the individual in need thereof is of Asian or African descent, and/or the individual in need thereof has an elevated level in a target tissue of one or more biomarkers selected from the group consisting of TGFβ1 expression; TGFβ2 expression; CTGF expression; phosphorylated cofilin; phosphorylated HSP27; and α-smooth muscle actin expression.

### DETAILED DESCRIPTION OF THE INVENTION

The single letter designation for amino acids is used predominately herein. As is well known by one of skill in the art, such single letter designations are as follows:

A is alanine; C is cysteine; D is aspartic acid; E is glutamic acid; F is phenylalanine; G is glycine; H is histidine; I is isoleucine; K is lysine; L is leucine; M is methionine; N is asparagine; P is proline; Q is glutamine; R is arginine; S is serine; T is threonine; V is valine; W is tryptophan; and Y is tyrosine.

As used herein, the singular forms “a”, “an” and “the” include plural referents unless the context clearly dictates otherwise. For example, reference to a “polypeptide” means one or more polypeptides.

The disclosure herein demonstrates that the polypeptides for use in the methods of the invention decrease transforming growth factor β1 (TGF-β1)-induced connective tissue growth factor (CTGF) expression and reduces the associated collagen synthesis. The effect was associated with changes in cell morphology (stellate morphology and disruption of stress fibers). Since the actin cytoskeleton should be intact for CTGF expression, this indicates that the ability of the polypeptides of the invention to alter cytoskeletal dynamics has important implications for the reduction of CTGF levels in keloid fibroblasts. Since CTGF plays a central role in the development and maintenance of the fibrotic response, the methods of the invention are broadly applicable for treating keloids and a wide range of fibrotic disorders.

Thus, in one aspect, the present invention provides methods for treating and/or limiting fibrotic disorders and/or scars selected from the group consisting of keloids and hypertrophic scars, comprising administering to an individual in need thereof an amount effective to treat and/or limit fibrotic disorders and/or scars selected from the group consisting of keloids and hypertrophic scars of a polypeptide comprising or consisting of a sequence according general formula I:

X1-A(X2)APLP-X3 (SEQ ID NO: 302 and SEQ ID NO: 316)

wherein X1 is 0-14 amino acids of the sequence of heat shock protein 20 between residues 1 and 14 of SEQ ID NO: 298;

X2 is selected from the group consisting of S, T, Y, D, E, hydroxylysine, hydroxyproline, phosphoserine analogs, and phosphotyrosine analogs; and

X3 is selected from the group consisting of (a) 0-140 amino acids of residues 21 and 160 of SEQ ID NO:298; and (b) 0, 1, 2, or 3 amino acids of a sequence of genus Z1-Z2-Z3, wherein Z1 is selected from the group consisting of G and D;

Z2 is selected from the group consisting of L and K; and

Z3 is selected from the group consisting of S, T, and K.

In a preferred embodiment, X1 is WLRR (SEQ ID NO: 1); Z1 is G; Z2 is L; and Z3 is K. In this embodiment, is thus preferred that the polypeptide of the general formula comprises or consists of an amino acid sequence according to SEQ ID NO: 300 (WLRRApSAPLPGLK), wherein the “pS” represents a phosphorylated serine residue. In a further preferred embodiment, the polypeptide for use in the methods of the invention comprises or consists of an amino acid sequence according to the formula:

B1-WLRRApSAPLPGLK-B2 (SEQ ID NO: 317),

wherein at least one of B1 and B2 are selected from the group consisting of YARAAARQARA (SEQ ID NO: 281) and YGRKKRRQRRR (SEQ ID NO: 299).

In one example, an “individual in need thereof” is an individual that has suffered or will suffer (for example, via a surgical procedure) a wound that may result in scar formation selected from the group consisting of keloids and hypertrophic scars and/or a fibrotic disorder, or has resulted in scar formation selected from the group consisting of keloids and hypertrophic scars and/or a fibrotic disorder. As used herein, the term “wound” refers broadly to injuries to the skin and subcutaneous tissue. Such wounds include, but are not limited to lacerations; burns; punctures; pressure sores; bed sores; canker sores; trauma, bites; fistulas; ulcers; lesions caused by infections; periodontal wounds; endodontic wounds; burning mouth syndrome; laparotomy wounds; surgical wounds; incisional wounds; contractures after burns; and wounds resulting from cosmetic surgical procedures.

As used herein, a “keloid” is a scar that results in an overgrowth of tissue at the site of a healed skin injury. Keloids are usually accompanied by severe itchiness, sharp pains and changes in texture. In severe cases, it can affect movement of skin. As used herein, “hypertrophic scars” are raised scars that do not grow beyond the boundaries of the original wound and may reduce over time.

As used herein, the phrase “reducing scar formation selected from the group consisting of keloids and hypertrophic scars” means any decrease in keloid or hypertrophic scar formation that provides a therapeutic or cosmetic benefit to the patient. Such a therapeutic or cosmetic benefit can be achieved, for example, by decreasing the size and/or depth of a keloid or hypertrophic scar relative to keloid or hypertrophic scar formation in the absence of treatment with the methods of the invention, or by reducing the size of an existing keloid or hypertrophic scar.

The present invention, by providing methods for reducing scar formation selected from the group consisting of keloids and hypertrophic scars, will be clinically useful for treating all types of wounds to reduce keloid and hypertrophic scar formation, both for reducing initial keloid or hypertrophic scar formation, and for therapeutic treatment of existing keloids or hypertrophic scars (i.e.: cutting out the keloid or hypertrophic scar after its formation, treating it with the compounds of the invention, and letting the keloid or hypertrophic scar heal more slowly).

In a preferred embodiment, individuals in need of treatment or limiting of scars selected from the group consisting of keloids and hypertrophic scars are highly pigmented individuals, including but not limited to individuals of Asian or African descent, that are susceptible to keloids and hypertrophic scars, and thus can benefit from the methods of the invention for prophylactic therapy to limit development of keloids or hypertrophic scars, as well as for treating keloids or hypertrophic scars.

In various other preferred embodiments, individuals in need of therapy for treating or limiting fibrotic disorders are those suffering from or at risk of one or more fibrotic disorders associated with TGFβ-induced CTGF expression, including but not limited to tissue fibrosis (including but not limited to idiopathic pulmonary fibrosis, hepatic fibrosis, renal fibrosis, retroperitoneal fibrosis, cystic fibrosis, blood vessel fibrosis and heart tissue fibrosis); diabetic nephropathy, glomerulosclerosis, and IgA nephropathy (causes of kidney failure and the need for dialysis and retransplant); diabetic retinopathy and macular degeneration (fibrotic diseases of the eye and leading causes of blindness); cirrhosis and biliary atresia (leading causes of liver fibrosis and failure); congestive heart failure; lung fibrosis; scleroderma; abdominal adhesions; and interstitial fibrosis.

In various other preferred embodiments of all of the embodiments disclosed herein, individuals in need of therapy for treating and/or limiting fibrotic disorders and/or scars selected from the group consisting of keloids and hypertrophic scars are those with elevated levels of one or more of the following biomarkers:

Transforming growth factor beta 1 (“TGFβ1”) expression;

Transforming growth factor beta 2 (“TGFβ2”) expression;

Connective tissue growth factor (“CTGF”) expression;

Phosphorylated cofilin;

Phosphorylated HSP27; and

α-smooth muscle actin expression.

As disclosed below, the polypeptides of the invention inhibit TGFβ1-induced CTGF expression, TGFβ1-induced expression of α-SMA and phosphorylation of cofilin and HSP27 in human keloid fibroblasts, all of which are elevated in fibrotic conditions, indicating that individuals with elevated levels of these biomarkers can especially benefit from the methods of the present invention. As used herein, an “elevated” level of the one or more biomarkers means any increase above normal for that individual or similarly situated individuals in a relevant target tissue. Such target tissues are those affected by fibrotic conditions, including but not limited to blood, wound exudate, and biopsies taken from tissues affected by fibrosis including but not limited to those disclosed above (skin, kidney, lung, liver, peritoneum, blood vessel, heart, retina, etc.) In various further embodiments, an individual in need thereof is one that has a level of one or more of the recited biomarkers 5%, 10%, 15%, 20%, 25%, 50%, 75%, 100%, or more above normal levels. Determining the level of the one or more biomarkers can be done using standard techniques in the art for measuring protein and/or gene expression, including but not limited to those disclosed below.

A “normal” level of these one or more biomarkers may be established by any suitable means, including but not limited to determining a normal level in that individual or similarly situated individuals in the absence of fibrotic conditions and/or keloids, or any other suitable means to establish a standard for reference.

As used herein, “treat” or “treating” means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

As used herein, the term “limit” or “limiting” means to limit the disorder in individuals at risk of developing the disorder.

In a further aspect, the present invention provides methods to monitor effectiveness of the treatment methods of the invention, comprising treating the individual as disclosed herein, and subsequently determining levels of one or more of the following biomarkers:

TGFβ1 expression;

TGFβ2 expression;

CTGF expression;

Phosphorylated cofilin;

Phosphorylated HSP27; and

α-smooth muscle actin expression.

In these embodiments, it is preferred that the level of the one or more biomarkers is determined prior to treatment to establish a pre-treatment level, followed by determining the biomarker levels post-treatment. The timing for such subsequent biomarker level determinations can be any that are deemed useful by an attending physician (ie: once per week following treatment; twice per week; once every other week, etc.). While efficacy of the treatment can be established by effect on the symptoms experienced by the individual, monitoring of the biomarker levels can provide additional information on the efficacy of treatment that is beneficial to an attending physician in determining a treatment regimen to pursue. For example, if the treatment regimen has not yet produced a noticeable improvement in the individual's symptoms but the biomarker levels indicate that the treatment is reducing the biomarker levels, then the physician may decide to continue the treatment regimen at the same dosage and frequency. Alternatively, if neither the symptoms or the biomarker levels are being impacted by the treatment, the attending physician may decide to increase the dosage and/or frequency, or to pursue a combination treatment (including, but not limited to, TGF-β antibody therapy, and/or therapies designed to inhibit α-smooth muscle actin expression and/or dephosphorylate HSP27 and/or cofilin).

Referring back to the general formula, residues 15-21 from HSP20, with possible substitutions at residue 16 of HSP20 form the structural core of the polypeptides according to general formula I (A(X2)APLP) (SEQ ID NO: 2). The full sequence of HSP20 is provided as SEQ ID NO: 298, and is shown below:

Met Glu Ile Pro Val Pro Val Gln Pro Ser Trp Leu
Arg Arg Ala Ser Ala Pro Leu Pro Gly Leu Ser Ala
Pro Gly Arg Leu Phe Asp Gln Arg Phe Gly Glu Gly
Leu Leu Glu Ala Glu Leu Ala Ala Leu Cys Pro Thr
Thr Leu Ala Pro Tyr Tyr Leu Arg Ala Pro Ser Val
Ala Leu Pro Val Ala Gln Val Pro Thr Asp Pro Gly
His Phe Ser Val Leu Leu Asp Val Lys His Phe Ser
Pro Glu Glu Ile Ala Val Lys Val Val Gly Glu His
Val Glu Val His Ala Arg His Glu Glu Arg Pro Asp
Glu His Gly Phe Val Ala Arg Glu Phe His Arg Arg
Tyr Arg Leu Pro Pro Gly Val Asp Pro Ala Ala Val
Thr Ser Ala Leu Ser Pro Glu Gly Val Leu Ser Ile
Gln Ala Ala Pro Ala Ser Ala Gln Ala Pro Pro Pro
Ala Ala Ala Lys.

The underlined residues represent amino acids 15-21.

X1 is 0-14 amino acids of SEQ ID NO: 298 between residues 1 and 14 of SEQ ID NO:298 (shown in italics above). Thus, if X1 is 5 amino acids of residues 1 and 14 of SEQ ID NO:298, then X1 would be the 5 amino acids contiguous to residues 15-21, eg: SWLRR (SEQ ID NO:303). Similarly, where X1 is the following number of amino acids of residues 1-14 of SEQ ID NO:298, its identity is as shown below:

| | | | |
|---|---|---|---|
| 1 | amino acid of SEQ ID NO: 298: | R | |
| 2 | amino acids of SEQ ID NO: 298: | RR | |
| 3 | amino acids of SEQ ID NO: 298: | LRR | (SEQ ID NO: 304) |
| 4 | amino acids of SEQ ID NO: 298: | WLRR | (SEQ ID NO: 1) |
| 6 | amino acids of SEQ ID NO: 298: | PSWLRR | (SEQ ID NO: 305) |
| 7 | amino acids of SEQ ID NO: 298: | NPSWLRR | (SEQ ID NO: 306) |
| 8 | amino acids of SEQ ID NO: 298: | VNPSWLRR | (SEQ ID NO: 307) |
| 9 | amino acids of SEQ ID NO: 298: | PVNPSWLRR | (SEQ ID NO: 308) |
| 10 | amino acids of SEQ ID NO: 298: | VPVNPSWLRR | (SEQ ID NO: 309) |
| 11 | amino acids of SEQ ID NO: 298: | PVPVNPSWLRR | (SEQ ID NO: 310) |
| 12 | amino acids of SEQ ID NO: 298: | IPVPPVNPSWLRR | (SEQ ID NO: 311) |
| 13 | amino acids of SEQ ID NO: 298: | EIPVPPVNPSWLRR | (SEQ ID NO: 312) |
| 14 | amino acids of SEQ ID NO: 298: | MEIPVPPVNPSWLRR | (SEQ ID NO: 313) |

In a further embodiment, X1 is 0, 1, 2, 3, or 4 amino acids of the sequence WLRR (SEQ ID NO:1).

In another embodiment, X3 is 0-140 amino acids between residues 21 and 160 of SEQ ID NO:298. According to this embodiment, X3 can be 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 120, 121, 122, 123, 124, 125, 126, 127, 128, 129, 130, 131, 132, 133, 134, 135, 136, 137, 138, 139 or 140 amino acids between residues 21 and 160 of SEQ ID NO:298.

For example, if X3 is 5 amino acids between residues 21 and 160 of SEQ ID NO:298, then X3 would be the 5 amino acids contiguous to residues 15-21, eg: GLSAP (SEQ ID NO: 314). Other possible X3 sequences will be apparent to one of skill in the art based on the teachings provided herein.

In another embodiment, X3 is 0, 1, 2, or 3 amino acids of a sequence of genus Z1-Z2-Z3, wherein Z1 is selected from the group consisting of G and D;

Z2 is selected from the group consisting of L and K; and

Z3 is selected from the group consisting of S, T, and K.

For example, if X3 is 2 amino acids of a sequence of the genus Z1-Z2-Z3, then the possibilities for X3 are GL, GK, DL, and DK. Other possible X3 sequences in this embodiment will be apparent to one of skill in the art based on the teachings provided herein.

According to various embodiments of the polypeptides of general formula I, X2 is S, T, Y, D E, a phosphoserine mimic, or a phosphotyrosine mimic. It is preferred that X2 is S, T, or Y; more preferred that X2 is S or T, and most preferred that X2 is S. In these embodiments where X2 is S, T, or Y, it is most preferred that X2 is phosphorylated. When X2 is D or E, these residues have a negative charge that mimics the phosphorylated state. The polypeptides of general formula I are optimally effective in the methods of the invention when X2 is phosphorylated, is a phosphoserine or phosphotyrosine mimic, or is another mimic of a phosphorylated amino acid residue, such as a D or E residue. Examples of phosphoserine mimics include, but are not limited to, sulfoserine, amino acid mimics containing a methylene substitution for the phosphate oxygen, 4-phosphono(difluoromethyl)phenylanaline, and L-2-amino-4-(phosphono)-4,4-difluorobutanoic acid. Other phosphoserine mimics can be made by those of skill in the art. Examples of phosphotyrosine mimics include, but are not limited to, phosphonomethylphenylalanine, difluorophosphonomethylphenylalanine, fluoro-O-malonyltyrosine and O-malonyltyrosine.

Thus, according to these various embodiments, a representative sample of polypeptides according to general formula I for use in the methods of the invention include, but are not limited to, polypeptides comprising or consisting of the following sequences: (ASAPLP) (SEQ ID NO:3); (ATAPLP) (SEQ ID NO:4); (RASAPLP) (SEQ ID NO:5); (RATAPLP) (SEQ ID NO:6); (AYAPLP) (SEQ ID NO:7); (RAYAPLP) (SEQ ID NO:8); (RRASAPLP) (SEQ ID NO:9); (LRRASAPLP) (SEQ ID NO:10); (WLRRASAPLP); (SEQ ID NO:11) (RRATAPLP) (SEQ ID NO:12); (LRRATAPLP) (SEQ ID NO:13); (WLRRATAPLP) (SEQ ID NO:14); (RRAYAPLP) (SEQ ID NO:15); (LRRAYAPLP) (SEQ ID NO:16); (WLRRAYAPLP) (SEQ ID NO:17); (RRASAPLPG) (SEQ ID NO:18); (RRASAPLPD) (SEQ ID NO:19); (RRASAPLPGL) (SEQ ID NO:20); (RRASAPLPGK) (SEQ ID NO:21); (RRASAPLPDL) (SEQ ID NO:22); (RRASAPLPDK) (SEQ ID NO:23); (RRASAPLPGLS) (SEQ ID NO:24); (RRASAPLPGLT) (SEQ ID NO:25); (RRASAPLPGKS) (SEQ ID NO:26); (RRASAPLPGKT) (SEQ ID NO:27); (RRASAPLPDLS) (SEQ ID NO:28); RRASAPLPDLT) (SEQ ID NO:29); (RRASAPLPDKS) (SEQ ID NO:30); (RRASAPLPDKT) (SEQ ID NO:31); (LRRASAPLPG) (SEQ ID NO:32); (LRRASAPLPD) (SEQ ID NO:33); (LRRASAPLPGL) (SEQ ID NO:34); (LRRASAPLPGK) (SEQ ID NO:35); (LRRASAPLPDL) (SEQ ID NO:36); (LRRASAPLPDK) (SEQ ID NO:37); (LRRASAPLPGLS) (SEQ ID NO:38); (LRRASAPLPGLT) (SEQ ID NO:39); (LRRASAPLPGKS) (SEQ ID NO:40); (LRRASAPLPGKT) (SEQ ID NO:41); (LRRASAPLPDLS) (SEQ ID NO:42); (LRRASAPLPDLT) (SEQ ID NO:43); (LRRASAPLPDKS) (SEQ ID NO:44); (LRRASAPLPDKT) (SEQ ID NO:45); (WLRRASAPLPG) (SEQ ID NO:46); (WLRRASAPLPD) (SEQ ID NO:47); (WLRRASAPLPGL) (SEQ ID NO:48); (WLRRASAPLPGK) (SEQ ID NO:49); (WLRRASAPLPDL) (SEQ ID NO:50); (WLRRASAPLPDK) (SEQ ID NO:51); (WLRRASAPLPGLS) (SEQ ID NO:52); (WLRRASAPLPGLT) (SEQ ID NO:53); (WLRRASAPLPGKS) (SEQ ID NO:54); (WLRRASAPLPGKT) (SEQ ID NO:55); (WLRRASAPLPDLS) (SEQ ID NO:56); (WLRRASAPLPDLT) (SEQ ID NO:57); (WLRRASAPLPDKS) (SEQ ID NO:58); (WLRRASAPLPDKT) (SEQ ID NO:59); (RRATAPLPG) (SEQ ID NO:60); (RRATAPLPD) (SEQ ID NO:61); (RRATAPLPGL) (SEQ ID NO:62); (RRATAPLPGK) (SEQ ID NO:63); (RRATAPLPDL) (SEQ ID NO:64); (RRATAPLPDK) (SEQ ID NO:65); (RRATAPLPGLS) (SEQ ID NO:66); (RRATAPLPGLT) (SEQ ID NO:67); (RRATAPLPGKS) (SEQ ID NO:68); (RRATAPLPGKT) (SEQ ID NO:69); (RRATAPLPDLS) (SEQ ID NO:70); (RRATAPLPDLT) (SEQ ID NO:71); (RRATAPLPDKS) (SEQ ID NO:72); (RRATAPLPDKT) (SEQ ID NO:73); (LRRATAPLPG) (SEQ ID NO:74); (LRRATAPLPD) (SEQ ID NO:75); (LRRATAPLPGL) (SEQ ID NO:76); (LRRATAPLPGK) (SEQ ID NO:77); (LRRATAPLPDL) (SEQ ID NO:78); (LRRATAPLPDK) (SEQ ID NO:79); (LRRATAPLPGLS) (SEQ ID NO:80); (LRRATAPLPGLT) (SEQ ID NO:81); (LRRATAPLPGKS) (SEQ ID NO:82); (LRRATAPLPGKT) (SEQ ID NO:83); (LRRATAPLPDLS) (SEQ ID NO:84); (LRRATAPLPDLT) (SEQ ID NO:85); (LRRATAPLPDKS) (SEQ ID NO:86); (LRRATAPLPDKT) (SEQ ID NO:87); (WLRRATAPLPG) (SEQ ID NO:88); (WLRRATAPLPD) (SEQ ID NO:89); (WLRRATAPLPGL) (SEQ ID NO:90); (WLRRATAPLPGK) (SEQ ID NO:91); (WLRRATAPLPDL) (SEQ ID NO:92); (WLRRATAPLPDK) (SEQ ID NO:93); (WLRRATAPLPGLS) (SEQ ID NO:94); (WLRRATAPLPGLT) (SEQ ID NO:95); (WLRRATAPLPGKS) (SEQ ID NO:96); (WLRRATAPLPGKT) (SEQ ID NO:97); (WLRRATAPLPDLS) (SEQ ID NO:98); (WLRRATAPLPDLT) (SEQ ID NO:99); (WLRRATAPLPDKS) (SEQ ID NO:100); (WLRRATAPLPDKT) (SEQ ID NO:101); (RRAYAPLPG) (SEQ ID NO:102); (RRAYAPLPD) (SEQ ID NO:103); (RRAYAPLPGL) (SEQ ID NO:104); (RRAYAPLPGK) (SEQ ID NO:105); (RRAYAPLPDL) (SEQ ID NO:106); (RRAYAPLPDK) (SEQ ID NO:107); (RRAYAPLPGLS) (SEQ ID NO:108); (RRAYAPLPGLT) (SEQ ID NO:109); (RRAYAPLPGKS) (SEQ ID NO:110; (RRAYAPLPGKT) (SEQ ID NO:111); (RRAYAPLPDLS) (SEQ ID NO:112); (RRAYAPLPDLT) (SEQ ID NO:113); (RRAYAPLPDKS) (SEQ ID NO:114); (RRAYAPLPDKT) (SEQ ID NO:115); (LRRAYAPLPG) (SEQ ID NO:116); (LRRAYAPLPD) (SEQ ID NO:117); (LRRAYAPLPGL) (SEQ ID NO:118); (LRRAYAPLPGK) (SEQ ID NO:119); (LRRAYAPLPDL) (SEQ ID NO:120); (LRRAYAPLPDK) (SEQ ID NO:121); (LRRAYAPLPGLS) (SEQ ID NO:122); (LRRAYAPLPGLT) (SEQ ID NO:123); (LRRAYAPLPGKS) (SEQ ID NO:124); (LRRAYAPLPGKT) (SEQ ID NO:125); (LRRAYAPLPDLS) (SEQ ID NO:126); (LRRAYAPLPDLT) (SEQ ID NO:127); (LRRAYAPLPDKS) (SEQ ID NO:128); (LRRAYAPLPDKT) (SEQ ID NO:129); (WLRRAYAPLPG) (SEQ ID NO:130); (WLRRAYAPLPD) (SEQ ID NO:131); (WLRRAYAPLPGL) (SEQ ID NO:132); (WLRRAYAPLPGK) (SEQ ID NO:133); (WLRRAYAPLPDL) (SEQ ID NO:134); (WLRRAYAPLPDK) (SEQ ID NO:135); (WLRRAYAPLPGLS) (SEQ ID NO:136); (WLRRAYAPLPGLT) (SEQ ID NO:137); (WLRRAYAPLPGKS) (SEQ ID NO:138); (WLRRAYAPLPGKT) (SEQ ID NO:139); (WLRRAYAPLPDLS) (SEQ ID NO:140); (WLRRAYAPLPDLT) (SEQ ID NO:141); (WLRRAYAPLPDKS) (SEQ ID NO:142); and (WLRRAYAPLPDKT) (SEQ ID NO:143); ((F/Y/W)RRASAPLP) (SEQ ID NO:144); ((F/Y/W)LRRASAPLP) (SEQ ID NO:145); ((F/Y/W)WLRRASAPLP); (SEQ ID NO:146) ((F/Y/W)RRATAPLP) (SEQ ID NO:147); ((F/Y/W)LRRATAPLP) (SEQ ID NO:148); ((F/Y/W)WLRRATAPLP) (SEQ ID NO:149); ((F/Y/W)RRAYAPLP) (SEQ ID NO:150); ((F/Y/W)LRRAYAPLP) (SEQ ID NO:151); ((F/Y/W)WLRRAYAPLP) (SEQ ID NO:152); ((F/Y/W)RRASAPLPG) (SEQ ID NO:153); ((F/Y/W)RRASAPLPD) (SEQ ID NO:154); ((F/Y/W)RRASAPLPGL) (SEQ ID NO:155); ((F/Y/W)RRASAPLPGK) (SEQ ID NO:156); ((F/Y/W)RRASAPLPDL) (SEQ ID NO:157); ((F/Y/W)RRASAPLPDK)_{] (SEQ ID NO:}158); ((F/Y/W)RRASAPLPGLS) (SEQ ID NO:159); ((F/Y/W)RRASAPLPGLT) (SEQ ID NO:160); ((F/Y/W)RRASAPLPGKS); (SEQ ID NO:161); ((F/Y/W)RRASAPLPGKT) (SEQ ID NO:162); ((F/Y/W)RRASAPLPDLS) (SEQ ID NO:163); ((F/Y/W)RRASAPLPDLT) (SEQ ID NO:164); ((F/Y/W)RRASAPLPDKS) (SEQ ID NO:165); ((F/Y/W)RRASAPLPDKT) (SEQ ID NO:166); ((F/Y/W)LRRASAPLPG) (SEQ ID NO:167); ((F/Y/W)LRRASAPLPD) (SEQ ID NO:168); ((F/Y/W))LRRASAPLPGL) (SEQ ID NO:169); ((F/Y/W)LRRASAPLPGK) (SEQ ID NO:170); ((F/Y/W)LRRASAPLPDL) (SEQ ID NO:171); ((F/Y/W)LRRASAPLPDK) (SEQ ID NO:172); ((F/Y/W)LRRASAPLPGLS) (SEQ ID NO:173); ((F/Y/W)LRRASAPLPGLT) (SEQ ID NO:174); ((F/Y/W)LRRASAPLPGKS) (SEQ ID NO:175); ((F/Y/W)LRRASAPLPGKT) (SEQ ID NO:176); ((F/Y/W)LRRASAPLPDLS) (SEQ ID NO:177); ((F/Y/W)LRRASAPLPDLT) (SEQ ID NO:178); ((F/Y/W)LRRASAPLPDKS) (SEQ ID NO:179); ((F/Y/W)LRRASAPLPDKT) (SEQ ID NO:180); ((F/Y/W)WLRRASAPLPG) (SEQ ID NO:181); ((F/Y/W)WLRRASAPLPD) (SEQ ID NO:182); ((F/Y/W)WLRRASAPLPGL) (SEQ ID NO:183); ((F/Y/W)WLRRASAPLPGK) (SEQ ID NO:184); ((F/Y/W)WLRRASAPLPDL) (SEQ ID NO:185); ((F/Y/W)WLRRASAPLPDK) (SEQ ID NO:186); ((F/Y/W)WLRRASAPLPGLS) (SEQ ID NO:187); ((F/Y/W)WLRRASAPLPGLT) (SEQ ID NO:188); ((F/Y/W)WLRRASAPLPGKS) (SEQ ID NO:189); ((F/Y/W)WLRRASAPLPGKT) (SEQ ID NO:190); ((F/Y/W)WLRRASAPLPDLS) (SEQ ID NO:191); ((F/Y/W)WLRRASAPLPDLT) (SEQ ID NO:192); ((F/Y/W)WLRRASAPLPDKS) (SEQ ID NO:193); ((F/Y/W)WLRRASAPLPDKT) (SEQ ID NO:194); ((F/Y/W)RRATAPLPG) (SEQ ID NO:195); ((F/Y/W)RRATAPLPD) (SEQ ID NO:196); ((F/Y/W)RRATAPLPGL) (SEQ ID NO:197); ((F/Y/W)RRATAPLPGK) (SEQ ID NO:198); ((F/Y/W)RRATAPLPDL) (SEQ ID NO:199); ((F/Y/W)RRATAPLPDK) (SEQ ID NO:200); ((F/Y/W)RRATAPLPGLS) (SEQ ID NO:201); ((F/Y/W)RRATAPLPGLT) (SEQ ID NO:202); ((F/Y/W)RRATAPLPGKS) (SEQ ID NO:203); ((F/Y/W)RRATAPLPGKT) (SEQ ID NO:204); ((F/Y/W)RRATAPLPDLS) (SEQ ID NO:205); ((F/Y/W)RRATAPLPDLT) (SEQ ID NO:206); ((F/Y/W)RRATAPLPDKS) (SEQ ID NO:207); ((F/Y/W)RRATAPLPDKT) (SEQ ID NO:208); ((F/Y/W)LRRATAPLPG) (SEQ ID NO:209); ((F/Y/W)LRRATAPLPD) (SEQ ID NO:210); ((F/Y/W)LRRATAPLPGL) (SEQ ID NO:211); ((F/Y/W)LRRATAPLPGK) (SEQ ID NO:212); ((F/Y/W)LRRATAPLPDL) (SEQ ID NO:213); ((F/Y/W)LRRATAPLPDK) (SEQ ID NO:214); ((F/Y/W)LRRATAPLPGLS) (SEQ ID NO:215); ((F/Y/W)LRRATAPLPGLT) (SEQ ID NO:216); ((F/Y/W)LRRATAPLPGKS) (SEQ ID NO:217); ((F/Y/W)LRRATAPLPGKT) (SEQ ID NO:218); ((F/Y/W)LRRATAPLPDLS) (SEQ ID NO:219); ((F/Y/W)LRRATAPLPDLT) (SEQ ID NO:220); ((F/Y/W)LRRATAPLPDKS) (SEQ ID NO:221); ((F/Y/W)LRRATAPLPDKT) (SEQ ID NO:222); ((F/Y/W)WLRRATAPLPG) (SEQ ID NO:223); ((F/Y/W)WLRRATAPLPD) (SEQ ID NO:224); ((F/Y/W)WLRRATAPLPGL) (SEQ ID NO:225); ((F/Y/W)WLRRATAPLPGK) (SEQ ID NO:226); ((F/Y/W)WLRRATAPLPDL) (SEQ ID NO:227); ((F/Y/W)WLRRATAPLPDK) (SEQ ID NO:228); ((F/Y/W)WLRRATAPLPGLS) (SEQ ID NO:229); ((F/Y/W)WLRRATAPLPGLT) (SEQ ID NO:230); ((F/Y/W)WLRRATAPLPGKS) (SEQ ID NO:231); ((F/Y/W)WLRRATAPLPGKT) (SEQ ID NO:232); ((F/Y/W)WLRRATAPLPDLS) (SEQ ID NO:233); ((F/Y/W)WLRRATAPLPDLT) (SEQ ID NO:234); ((F/Y/W)WLRRATAPLPDKS) (SEQ ID NO:235); ((F/Y/W)WLRRATAPLPDKT) (SEQ ID NO:236); ((F/Y/W)RRAYAPLPG) (SEQ ID NO:237); ((F/Y/W)RRAYAPLPD) (SEQ ID NO:238); ((F/Y/W)RRAYAPLPGL) (SEQ ID NO:239); ((F/Y/W)RRAYAPLPGK) (SEQ ID NO:240); ((F/Y/W)RRAYAPLPDL) (SEQ ID NO:241); ((F/Y/W)RRAYAPLPDK) (SEQ ID NO:242); ((F/Y/W)RRAYAPLPGLS) (SEQ ID NO:243); ((F/Y/W)RRAYAPLPGLT) (SEQ ID NO:244); ((F/Y/W)RRAYAPLPGKS) (SEQ ID NO:245); ((F/Y/W)RRAYAPLPGKT) (SEQ ID NO:246); ((F/Y/W)RRAYAPLPDLS) (SEQ ID NO:247); ((F/Y/W)RRAYAPLPDLT) (SEQ ID NO:248); ((F/Y/W)RRAYAPLPDKS) (SEQ ID NO:249); ((F/Y/W)RRAYAPLPDKT) (SEQ ID NO:250); ((F/Y/W)LRRAYAPLPG) (SEQ ID NO:251); ((F/Y/W)LRRAYAPLPD) (SEQ ID NO:252); ((F/Y/W)LRRAYAPLPGL) (SEQ ID NO:253); ((F/Y/W)LRRAYAPLPGK) (SEQ ID NO:254); ((F/Y/W)LRRAYAPLPDL) (SEQ ID NO:255); ((F/Y/W)LRRAYAPLPDK) (SEQ ID NO:256); ((F/Y/W)LRRAYAPLPGLS) (SEQ ID NO:257); ((F/Y/W)LRRAYAPLPGLT) (SEQ ID NO:258); ((F/Y/W)LRRAYAPLPGKS) (SEQ ID NO:259); ((F/Y/W)LRRAYAPLPGKT) (SEQ ID NO:260); ((F/Y/W)LRRAYAPLPDLS) (SEQ ID NO:261); ((F/Y/W)LRRAYAPLPDLT) (SEQ ID NO:262); ((F/Y/W)LRRAYAPLPDKS) (SEQ ID NO:263); ((F/Y/W)LRRAYAPLPDKT) (SEQ ID NO:264); ((F/Y/W)WLRRAYAPLPG) (SEQ ID NO:265); ((F/Y/W)WLRRAYAPLPD) (SEQ ID NO:266); ((F/Y/W)WLRRAYAPLPGL) (SEQ ID NO:267); ((F/Y/W)WLRRAYAPLPGK) (SEQ ID NO:268); ((F/Y/W)WLRRAYAPLPDL) (SEQ ID NO:269); ((F/Y/W)WLRRAYAPLPDK) (SEQ ID NO:270); ((F/Y/W)WLRRAYAPLPGLS) (SEQ ID NO:271); ((F/Y/W)WLRRAYAPLPGLT) (SEQ ID NO:272); ((F/Y/W)WLRRAYAPLPGKS) (SEQ ID NO:273); ((F/Y/W)WLRRAYAPLPGKT) (SEQ ID NO:274); ((F/Y/W)WLRRAYAPLPDLS) (SEQ ID NO:275); ((F/Y/W)WLRRAYAPLPDLT) (SEQ ID NO:276); ((F/Y/W)WLRRAYAPLPDKS) (SEQ ID NO:277); and ((F/Y/W)WLRRAYAPLPDKT) (SEQ ID NO:278) wherein (F/Y/W) means that the residue is selected from F, Y, and W. Other specific polypeptides falling within the scope of general formula I will be readily apparent to one of skill in the art based on the teachings herein.

The polypeptides of general formula I may be present in multiple copies to provide increased efficacy for use in the methods of the invention. For example, the polypeptides may be present in 1, 2, 3, 4, or 5 copies. In a further embodiment, the polypeptides comprising a sequence according to general formula I comprise a combination of different sequences from the region X1-A(X2)APLP-X3 (SEQ ID NO: 302 and SEQ ID NO: 316). In this embodiment, for example, the polypeptide can consist of 1 copy of SEQ ID NO: 9 and 1 copy of SEQ ID NO: 143. In a different example, the polypeptide could consist of 2 copies of SEQ ID NO: 200 and 3 copies of SEQ ID NO: 62. It will be apparent to one of skill in the art that many such combinations are possible based on the teachings of the present invention.

In a preferred embodiment, the polypeptides according to general formula I further comprise one or more transduction domains. As used herein, the term “transduction domain” means an amino acid sequence that can carry the polypeptide across cell membranes. These domains can be linked to other polypeptides to direct movement of the linked polypeptide across cell membranes. In some cases the transducing molecules do not need to be covalently linked to the active polypeptide. In a preferred embodiment, the transduction domain is linked to the rest of the polypeptide via peptide bonding. Examples of such transduction domains include, but are not limited to (R)₄₋₉ (SEQ ID NO:279); GRKKRRQRRRPPQ (SEQ ID NO:280); YARAAARQARA (SEQ ID NO:281); DAATATRGRSAASRPTERPRAPARSASRPRRPVE (SEQ ID NO:282); GWTLNSAGYLLGLINLKALAALAKKIL (SEQ ID NO:283); PLSSIFSRIGDP (SEQ ID NO:284); AAVALLPAVLLALLAP (SEQ ID NO:285); AAVLLPVLLAAP (SEQ ID NO:286); VTVLALGALAGVGVG (SEQ ID NO:287); GALFLGWLGAAGSTMGAWSQP (SEQ ID NO:288); GWTLNSAGYLLGLINLKALAALAKKIL (SEQ ID NO:289); KLALKLALKALKAALKLA (SEQ ID NO:290); KETWWETWWTEWSQPKKKRKV (SEQ ID NO:291); KAFAKLAARLYRKAGC (SEQ ID NO:292); KAFAKLAARLYRAAGC (SEQ ID NO:293); AAFAKLAARLYRKAGC (SEQ ID NO:294); KAFAALAARLYRKAGC (SEQ ID NO:295); KAFAKLAAQLYRKAGC (SEQ ID NO:296), GGGGYGRKKRRQRRR (SEQ ID NO:297), and YGRKKRRQRRR (SEQ ID NO:299).

In a further embodiment, the polypeptides comprise or consist of polypeptides of the formula:

B1-X1-A(X2)APLP-X3-B2 (SEQ ID NO: 318 and SEQ ID NO: 319)

wherein X1, X2, and X3 are as defined above, and wherein B1 and B2 are independently absent or comprise a transduction domain, as described above.

In a preferred embodiment, one or both of B1 and B2 comprise or consist of the amino acid sequence of YGRKKRRQRRR (SEQ ID NO:299) and/or YARAAARQARA (SEQ ID NO:281). In a most preferred embodiment, the polypeptide according to the general formulas disclosed herein comprises or consists of a polypeptide YGRKKRRQRRRWLRRApSAPLPGLK (SEQ ID NO:301) or YARAAARQARAWLRRApSAPLPGLK (SEQ ID NO:315), wherein “pS” represents a phosphorylated serine residue.

In a further embodiment of the methods of the present invention, the polypeptides comprise or consist of polypeptides of the formula:

J2-X1-A(X2)APLP-X3-J3 (SEQ ID NO: 320 and SEQ ID NO: 321)

wherein X1, X2, and X3 are as defined above, wherein J2 and J3 are independently absent or comprise a transduction domain, as described above.

The polypeptides for use in the methods of the invention can further be derivatized to provide enhanced half-life, for example, by linking to polyethylene glycol. The polypeptides of the invention may comprise L-amino acids, D-amino acids (which are resistant to L-amino acid-specific proteases in vivo), a combination of D- and L-amino acids, and various “designer” amino acids (e.g., β-methyl amino acids, Cα-methyl amino acids, and Nα-methyl amino acids, etc.) to convey special properties. Synthetic amino acids include ornithine for lysine, and norleucine for leucine or isoleucine.

In addition, the polypeptides can have peptidomimetic bonds, such as ester bonds, to prepare polypeptides with novel properties. For example, a peptide may be generated that incorporates a reduced peptide bond, i.e., R₁—CH₂—NH—R₂, where R₁ and R₂ are amino acid residues or sequences. A reduced peptide bond may be introduced as a dipeptide subunit. Such polypeptides are resistant to protease activity, and possess an extended half-live in vivo.

The term “polypeptide” is used in its broadest sense to refer to a sequence of subunit amino acids, amino acid analogs, or peptidomimetics. The subunits are linked by peptide bonds, although the polypeptide can comprise further moieties that are not necessarily linked to the polypeptide by a peptide bond. For example, as discussed above, the polypeptide can further comprise a non-amino acid molecule that contains an aromatic ring.

The polypeptides described herein may be chemically synthesized or recombinantly expressed. Recombinant expression can be accomplished using standard methods in the art, generally involving the cloning of nucleic acid sequences capable of directing the expression of the polypeptides into an expression vector, which can be used to transfect or transduce a host cell in order to provide the cellular machinery to carry out expression of the polypeptides. Such expression vectors can comprise bacterial or viral expression vectors, and such host cells can be prokaryotic or eukaryotic.

Preferably, the polypeptides for use in the methods of the present invention are chemically synthesized. Synthetic polypeptides, prepared using the well-known techniques of solid phase, liquid phase, or peptide condensation techniques, or any combination thereof, can include natural and unnatural amino acids. Amino acids used for peptide synthesis may be standard Boc (Nα-amino protected Nα-t-butyloxycarbonyl) amino acid resin with the standard deprotecting, neutralization, coupling and wash protocols of standard solid phase procedure, or base-labile Nα-amino protected 9-fluorenylmethoxycarbonyl (Fmoc) amino acids. Both Fmoc and Boc Nα-amino protected amino acids can be obtained from Sigma, Cambridge Research Biochemical, or other chemical companies familiar to those skilled in the art. In addition, the polypeptides can be synthesized with other Nα-protecting groups that are familiar to those skilled in this art.

Solid phase peptide synthesis may be accomplished by techniques familiar to those in the art and provided, for example by using automated synthesizers.

As used herein, an “amount effective” of the one or more polypeptides is an amount that is sufficient to provide the intended benefit of treatment. An effective amount of the polypeptides that can be employed ranges generally between about 0.01 μg/kg body weight and about 10 mg/kg body weight, preferably ranging between about 0.05 μg/kg and about 5 mg/kg body weight. However dosage levels are based on a variety of factors, including the type of injury, the age, weight, sex, medical condition of the individual, the severity of the condition, the route of administration, and the particular compound employed. Thus, the dosage regimen may vary widely, but can be determined routinely by a physician using standard methods.

The polypeptides may be subjected to conventional pharmaceutical operations such as sterilization and/or may contain conventional adjuvants, such as preservatives, stabilizers, wetting agents, emulsifiers, buffers etc.

For administration, the polypeptides are ordinarily combined with one or more adjuvants appropriate for the indicated route of administration. The compounds may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, dextran sulfate, heparin-containing gels, and/or polyvinyl alcohol, and tableted or encapsulated for conventional administration. Alternatively, the compounds of this invention may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

The polypeptides or pharmaceutical compositions thereof may be administered by any suitable route, including orally, parentally, by inhalation spray, rectally, or topically in dosage unit formulations containing conventional pharmaceutically acceptable carriers, adjuvants, and vehicles. The term parenteral as used herein includes, subcutaneous, intravenous, intra-arterial, intramuscular, intrasternal, intratendinous, intraspinal, intracranial, intrathoracic, infusion techniques or intraperitoneally. Preferred embodiments for administration vary with respect to the condition being treated. In a preferred embodiment, the polypeptides or pharmaceutical compositions are disposed on or in a wound dressing or other topical administration. Such wound dressings can be any used in the art, including but not limited to films (e.g., polyurethane films), hydrocolloids (hydrophilic colloidal particles bound to polyurethane foam), hydrogels (cross-linked polymers containing about at least 60% water), foams (hydrophilic or hydrophobic), calcium alginates (nonwoven composites of fibers from calcium alginate), cellophane, and biological polymers such as those described in US patent application publication number 20030190364, published Oct. 9, 2003.

The polypeptides may be made up in a solid form (including granules, powders or suppositories) or in a liquid form (e.g., solutions, suspensions, or emulsions). The polypeptides of the invention may be applied in a variety of solutions. Suitable solutions for use in accordance with the invention are sterile, dissolve sufficient amounts of the polypeptides, and are not harmful for the proposed application.

### Example 1

Keloids and hypertrophic scars are fibroproliferative abnormal healing disorders characterized by excessive scarring due to excessive production, deposition and contraction of extracellular matrix, which results in functional and cosmetic deformity (Leask and Abraham, 2004). There is no current effective treatment for these conditions.

One of the primary regulatory factors involved in initiating the wound-healing cascade is transforming growth factor (TGF)-β. There are three mammalian isoforms, designated TGF-β1, -β2 and -β3. TGF-β is a multifunctional molecule with effects that depend on the cellular context and balance of isoforms expressed. TGF-β1 is thought to be involved in the initiation of fibrotic response, whereas TGF-β3 may have anti-fibrotic functions (Leask and Abraham, 2004, Miller and Nanchahal, 2005).

In fibroblasts, TGF-β stimulates the expression of connective tissue growth factor (CTGF). CTGF is a cystein-rich peptide that acts as a downstream mediator of TGF-β, promoting fibroblast proliferation, extracellular matrix production (including collagen and fibronection) and granulation tissue formation (Duncan et al. 1999, Leask and Abraham, 2004). The expression of CTGF is also up-regulated by other compounds released upon tissue injury, such as thrombin and endothelin (Chambers et al., 2000, Shi-Wen et al., 2004, Rodriguez-Vita et al, 2005), thus suggesting that a complex network of cell-matrix-cytokine interactions regulate the initiation of the wound healing process as well as the pathological fibrotic disorders (Duncan et al., 1999). Because overexpression of CTGF in keloids and hypertrophic scars enhances the profibrotic response to TGF-β, CTGF is believed to participate in the pathogenesis of scar fibrosis (Colwell et al., 2004). Hence, it is conceivable that blockade of CTGF expression may attenuate the fibroproliferative response of pathological scarring by preventing connective tissue cell proliferation and matrix deposition.

In addition to cytokines and proteases, modifications in the cytoarchitecture affect the expression of CTGF. Accordingly, agents that disrupt microtubule, activate RhoA and stabilize actin fibers were demonstrated to increase CTGF expression in renal fibroblasts, whereas agents that led to actin depolymerization (such as latrunculin) decreased CTGF expression (Ott et al., 2003).

It was recently demonstrated that the P20 peptide (a phosphopeptide analogue to the heat shock protein 20) linked to a peptide carrier called protein transduction domain (PTD) penetrates into cells and inhibits stress fiber formation to stimulation with serum or lysophosphatidic acid (Dreiza et al., 2004). This led us to hypothesize that cytoskeleton disruption by the P20 peptide inhibits CTGF formation and may limit fibroproliferative conditions. To test this hypothesis, we investigated whether treatment with the PTD-P20 peptide would reduce the CTGF and collagen expression by TGF-β-stimulated keloid fibroblasts.

### Methods

Fibroblast culture: Human keloid fibroblasts were grown in 10 cm² dishes to 70% confluence in DMEM with 10% FBS and additional penicillin and streptomycin (1%), at 37° C. and 10% CO₂. Cells were serum starved in DMEM containing 0.5% FBS for 48 h before the experiment. At the start of the assay, fresh media was added to the dishes, and cells were either untreated (control) or treated with TGF-β1 (doses ranging from 0.6 to 5 ng/mL), P20 phosphopeptide (doses ranging from 50-200 μM), forskolin (FSK, 10 μM) or SNAP (500 μM) for 24 h. To verify the influence of serum starvation, we also had cells in 10% FBS containing medium (control high serum).

Western blot analysis: At the end of the experiment, cells were rinsed with PBS, and homogenized using UDC buffer. Lysates were mixed, centrifuged (6000×g) for 20 min, and the supernatant was used for determination of CTGF and collagen expression. Samples (20 μg of protein) were loaded on 15 or 10% SDS-PAGE gels, and the proteins were electrophoretically transferred to Immobilon membranes. To block non-specific binding, the membranes were incubated with 1:1(v/v) Tris-buffered saline (TBS):blocking buffer (Odyssey), stained with primary antibodies against CTGF (Torrey Pines) and collagen (Cortex) for 1 h at room temperature, and washed 3 times with TBS. Following, membranes were incubated with secondary anti-rabbit and anti-mouse antibodies, and washed with TBS containing Tween. Protein-antibody complexes were visualized using the Odyssey direct infrared fluorescence imaging system (Li-Cor, Lincoln, Nebr.).

Immunocytochemistry: Human keloid fibroblasts were grown on 6-well dishes with coverslips at 2.5×10⁵ cells/well. They were serum starved for 24 h, and then treated with the stimuli. Untreated (control) or cells treated with TGF-β1 (1.2 or 2.5 ng/mL) and/or P20 phosphopeptide (50 μM) were fixed with 4% paraformoldehyde, and permeabilized with 0.1% Triton X. Cells were then stained with Alexa 350 phalloidin to visualize actin filaments. Fluorescent images were obtained using a Zeiss microscope equipped with UV filter and Zeiss software.

Statistical analysis: All numerical data are presented as means±standard deviation from 3-6 experiments. ANOVA followed by Tukkey post-hoc test was used to compare experimental groups. The level of significance was set at p<0.05.

### Results

TGF-β1 and CTGF expression: Human keloid fibroblasts were serum-starved in DMEM medium containing 0.5% FBS for 48 hours, and treated with different doses of TGF-beta1 for 24 hours. CTGF expression was related to GAPDH expression by densitometry of western blots to correct for loading differences. The expression of CTGF in control cells was set to 1 for comparison of different blots.

The data demonstrated that TGF-β1 treatment for 24 h dose-dependently enhanced (2.1- to 4.6-fold) CTGF expression in human keloid fibroblasts.

P20 treatment and CTGF expression: Human keloid fibroblasts were serum-starved in DMEM medium containing 0.5% FBS for 48 hours, stimulated with TGF-beta1 doses ranging from 1.2 to 5 ng/mL and concomitantly treated with the P20 phosphopeptide (50, 100 or 200 μM) for 24 hours. Western blot bands were quantified by densitometry, and CTGF expression was related to GAPDH expression to correct for loading differences. The expression of CTGF in control cells was set to 1 for comparison of different blots. The data show that treatment with PTD-P20 significantly (p<0.05) reduced TGF-β1-induced CTGF expression in keloid fibroblasts. Reductions of 53% and 29% were observed for 1.2 and 2.5 ng/mL TGF-β1, respectively. On the other hand, when 5 ng/mL of TGF-β1 was used to stimulate the cells, PTD-P20 treatment did not reduce the CTGF expression even when used at higher doses (100 and 200 μM).

P20 treatment and collagen production: Having observed that PTD-P20 treatment reduced CTGF expression in cells stimulated with 1.2 and 2.5 ng/mL of TGF-β1, we next investigated whether collagen synthesis was also reduced. Human keloid fibroblasts were serum-starved in DMEM medium containing 0.5% FBS for 48 hours, stimulated with TGF-beta1 doses ranging from 1.2 to 2.5 ng/mL and concomitantly treated with the P20 phosphopeptide (50 μM) for 24 hours. Western blot bands were quantified by densitometry, and collagen expression was related to GAPDH expression to correct for loading differences. The expression of collagen in control cells was set to 1 for comparison of different blots. The data demonstrate that PTD-P20 treatment reduced collagen synthesis by ˜48%.

Treatment with compounds that elevate cAMP and cGMP: We next evaluated the influence of compounds that elevate cAMP (Forskolin, FSK) or cGMP (SNAP) on CTGF expression in keloid fibroblasts. Human keloid fibroblasts were serum-starved in DMEM medium containing 0.5% FBS for 48 hours, stimulated with TGF-beta1 dose of 2.5 ng/mL and concomitantly treated with the FSK (10 μM) for 24 hours. Western blot bands were quantified by densitometry, and CTGF expression was related to GAPDH expression to correct for loading differences. The expression of CTGF in control cells was set to 1 for comparison of different blots. Treatment of TGF-β1-stimulated fibroblasts (TGF-β1 dose of 2.5 ng/mL) with FSK resulted in a decrease of ˜50% of CTGF expression. No difference was observed in the CTGF expression when non-stimulated fibroblasts were treated with FSK compared to untreated cells. On the other hand, treatment of TGF-β1-stimulated cells with SNAP did not decrease CTGF expression. In addition, treatment of non-stimulated cells with SNAP resulted in a significant increase (p<0.05, two-time increase) in CTGF expression compared to untreated (control) cells. These results are in accordance with previous reports (Duncan et al., 1999) showing that the inhibitory effect on CTGF expression appeared to be selective for agents elevating cAMP. In addition, recent studies demonstrated that exposure of keloid fibroblasts to exogenous nitric oxide resulted in increased collagen expression in a dose-dependent manner (Hsu et al., 2006).

Influence of PTD-P20 treatment on the actin cytoskeleton of cells: Human keloid fibroblasts were serum-starved in DMEM medium containing 0.5% FBS for 48 hours, stimulated with TGF-beta1 (1.2 or 2.5 ng/mL) and/or P20 (50 μM) for 24 hours. The cells were then stained with phalloidin to evaluate the influence of PTD-P20 treatment on the actin cytoskeleton. PTD-P20 treatment led to stellate cell morphology and disrupted the actin cytoskeleton of non-stimulated cells and those stimulated with TGF-β1 at 1.2 ng/mL. This effect was less evident when TGF-β1 dose was 2.5 ng/mL.

### SUMMARY

Currently, there is no effective treatment for keloids and other fibrotic disorders. Most of the current investigated therapeutics target cell surface receptors or enzymes within the TGF-β signaling cascade. The approach presented here is innovative since it proposes a therapeutic use of a downstream protein in the kinase cascades that was identified by our group. The results presented herein demonstrate that PTD-P20 decreases TGF-β1-induced CTGF expression (at a level comparable to FSK) and reduces the associated collagen synthesis. The effect of PTD-P20 was associated with changes in cell morphology (stellate morphology and disruption of stress fibers). Since the actin cytoskeleton should be intact for CTGF expression, we suggest that the ability of PTD-P20 to alter cytoskeletal dynamics has important implications for the reduction of CTGF levels in keloid fibroblasts. Since CTGF plays a central role in the development and maintenance of the fibrotic response, the use of PTD-P20 represents a potential strategy to treat keloids and other fibrotic disorders.

### REFERENCES FOR EXAMPLE 1

Leask A, Abraham D J. FASEB J., 18(7):816-27, 2004.
Miller M C, Nanchahal J. BioDrugs, 19(6):363-81, 2005.
Duncan M R, et al. FASEB J., 13(13):1774-86, 1999.
Chambers et al. J Biol. Chem., 10; 275(45):35584-91, 2000.
Shi-Wen X, et al. Mol Biol Cell., 15(6):2707-19, 2004.
Rodriguez-Vita J et al. Circulation, 111(19):2509-17, 2005.
Colwell A S et al. Plast Reconstr Surg., 116(5):1387-90, 2005.
Ott C et al. J Biol. Chem., 278(45):44305-11, 2003.
Dreiza et al. FASEB J., 19(2):261-3, 2005.
Hsu et al. Nitric Oxide., 14(4):327-34, 2006.

### Example 2

Fibroblasts are widely recognized as a critical cell type involved in fibrosis, wound healing, and tissue repair. Less appreciated is the notion that the transformation of fibroblasts to myofibroblasts is a key, perhaps even essential, event for the cell to perform those functions (Powell, et al., 1999 and Tomasek, et al., 2002). Myofibroblasts are smooth muscle-like fibroblasts that express α-smooth muscle actin (α-SMA) and contain a contractile apparatus composed of actin filaments and associated proteins organized into prominent stress fibers (Tomasek, et al., 2002). In addition to their normal role in tissue homeostasis and repair, altered numbers and functions of myofibroblasts have been implicated in diseases with increased extracellular matrix (ECM) deposition and resultant fibrosis, such as those involving lung (pulmonary fibrosis), blood vessels (intimal hyperplasia), heart (cardiac fibrosis), and skin (keloids) (Desmouliere, et al., 2003, Desmouliere, et al., 2005, Hewitson, et al., 1995, Mitchell, et al, 1989, Zhang, et al., 1994, Naugle, et al., 2006, Chipev, et al., 2000, Pepper, et al., 1997, Heusinger-Ribeiro, et al., 2001). Thus, inhibition of the fibroblast-to-myofibroblast phenotypic modulation may provide a means to inhibit fibrosis in response to stimuli such as TGFβ1 and other mediators.

It was recently demonstrated that the P20 peptide (a phosphopeptide analogue to the heat shock protein 20) linked to a peptide carrier called protein transduction domain (PTD) penetrates into cells and inhibits stress fiber formation to stimulation with serum or lysophosphatidic acid (Dreiza et al., 2004). As discussed above, this peptide PTD-P20 also inhibits TGFβ1-induced CTGF expression in human keloid fibroblasts. In these experiments, the anti-fibrotic activity of PTD-P20 has been further examined by determining the effects on additional fibrotic molecules: α-SMA and the actin accessory proteins cofilin and HSP27. Cofilin is activated when dephosphorylated to depolymerize actin filaments, whereas HSP27 is activated upon phosphorylation and is associated with stress fiber formation. Thus, a fibrotic phenotype would be associated with increased phosphorylation of cofilin and HSP27. These results indicate that PTD-P20 inhibits TGFβ1-induced expression of α-SMA and phosphorylation of cofilin and HSP27. Such information adds to the understanding of the mechanism of action of PTD-P20 and identifies potential biomarkers that could be used to detect either activity of PTD-P20 or fibrotic disease state.

### Methods

Fibroblast culture: Human keloid fibroblasts were grown in 10 cm² dishes to 70% confluence in DMEM with 10% FBS and additional penicillin and streptomycin (1%), at 37° C. and 10% CO₂. Cells were serum starved in DMEM containing 0.5% FBS for 48 h before the experiment. At the start of the assay, fresh media was added to the dishes, and cells were either untreated (control) or treated with TGF-β1 (doses ranging from 0.6 to 5 ng/mL), P20 phosphopeptide (doses ranging from 50-200 μM), forskolin (FSK, 10 μM) or SNAP (500 μM) for 24 h. To verify the influence of serum starvation, we also had cells in 10% FBS containing medium (control high serum).

Western blot analysis: At the end of the experiment, cells were rinsed with PBS, and homogenized using UDC buffer. Lysates were mixed, centrifuged (6000×g) for 20 min, and the supernatant was used for determination of protein expression. Samples (20 μg of protein) were loaded on 15 or 10% SDS-PAGE gels, and the proteins were electrophoretically transferred to Immobilon membranes. To block non-specific binding, the membranes were incubated with 1:1(v/v) Tris-buffered saline (TBS):blocking buffer (Odyssey), stained with primary antibodies against α-smooth muscle actin expression, phosphorylated HSP27, and phosphorylated cofilin for 1 h at room temperature, and washed 3 times with TBS. Following, membranes were incubated with secondary anti-rabbit and anti-mouse antibodies, and washed with TBS containing Tween. Protein-antibody complexes were visualized using the Odyssey direct infrared fluorescence imaging system (Li-Cor, Lincoln, Nebr.).

Statistical analysis: All numerical data are presented as means±standard deviation from 3-6 experiments. ANOVA followed by Tukkey post-hoc test was used to compare experimental groups. The level of significance was set at p<0.05.

### Results

P20 treatment and α-smooth muscle actin expression: Human keloid fibroblasts were serum-starved for 48 hours in DMEM medium containing 0.5% FBS and treated with PTD-P20 (50 μM) with or without TGFβ1 (2.5 ng/mL) for 24 hours. Expression levels of α-SMA and beta-actin were quantified by densitometry of Western blots and were normalized to GAPDH expression to correct for loading differences. Protein expression in control cells was set to 1 for comparison of different blots. The data show that treatment with PTD-P20 significantly (p<0.05) reduced α-SMA expression in keloid fibroblasts with or without TGFβ1 treatment (FIG. 1). On the other hand, PTD-P20 had no effect on beta-actin expression, suggesting its activity is specific to α-SMA, a key fibrotic marker.

P20 treatment and HSP27 and cofilin phosphorylation: Human keloid fibroblasts were serum-starved in DMEM medium containing 0.5% FBS for 48 hours and treated with PTD-P20 (50 μM) with or without TGFβ1 (2.5 ng/mL) for 24 hours. Expression levels of phosphorylated cofilin and HSP27 were quantified by densitometry of Western blots and were normalized to GAPDH expression to correct for loading differences. Protein expression in control cells was set to 1 for comparison of different blots. The data demonstrate that treatment with PTD-P20 significantly (p<0.05) reduced TGFβ1-induced increases in cofilin and HSP27 phosphorylation in keloid fibroblasts. Levels of total cofilin and HSP27 (phosphorylated plus nonphosphorylated) did not change. These data suggest that PTD-P20 inhibits the TGFβ1 fibrotic response on many levels that impact the actin cytoskeleton.

### SUMMARY

The results presented herein demonstrate that PTD-P20 decreases TGFβ1-induced α-SMA expression and reduces the phosphorylation of cofilin and HSP27. Previous results have shown that PTD-P20 treatment was associated with changes in cell morphology (stellate morphology and disruption of stress fibers) and inhibition of CTGF expression. Since an intact actin cytoskeleton is important for CTGF expression, these data suggest that PTD-P20 inhibits fibrotic responses by altering cytoskeletal dynamics. Since the actin cytoskeleton plays a central role in the development and maintenance of the fibrotic response, the use of PTD-P20 represents a potential strategy to treat keloids and other fibrotic disorders. Taken together, these results also identify potential biomarkers (CTGF, α-SMA, cofilin, and HSP27) that could be used to detect either activity of PTD-P20 or fibrotic disease state.

The text file of the sequence listing submitted herewith, entitled “06-558-PCT_ST25.txt”, created Jul. 5, 2007, and 86,964 bytes in size, is incorporated herein by reference in its entirety.

### REFERENCES FOR EXAMPLE 2

Chipev et al. (2000) Cell Death Differ 7, 166-176
Desmouliere et al. (2003) Lab Invest 83, 1689-1707
Desmouliere et al. Wound Repair Regen 13, 7-12
Heusinger-Ribeiro et al. (2001) J Am 28-37
Hewitson et al. (1995) Am J Nephrol 15, 411-417
Mitchell et al. (1989) Lab Invest 60, 643-650
Naugle et al. (2006) Am J Physiol Heart Circ Physiol 290, H323-330
Pepper, M. S. (1997) Cytokine Growth Factor Rev 8, 21-43
Powell et al. (1999) Am J Physiol 277, C1-9
Tomasek et al. (2002) Nat Rev Mol Cell Biol 3, 349-363
Zhang et al. (1994) Am J Pathol 145, 114-125

We claim:

1. A method for treating and/or limiting fibrotic disorders comprising administering to an individual in need thereof an amount effective to treat and/or limit fibrotic disorders of a polypeptide comprising a sequence according to general formula I:
X1-A(X2)APLP-X3
wherein X1 is 0-14 amino acids of the sequence of heat shock protein 20 between residues 1 and 14 of SEQ ID NO: 298;
X2 is selected from the group consisting of S, T, Y, D, E, hydroxylysine, hydroxyproline, phosphoserine analogs, and phosphotyrosine analogs; and
X3 is selected from the group consisting of (a) 0-140 amino acids of residues 21 and 160 of SEQ ID NO:298; and (b) 0, 1, 2, or 3 amino acids of a sequence of genus Z1-Z2-Z3, wherein Z1 is selected from the group consisting of G and D;
Z2 is selected from the group consisting of L and K; and
Z3 is selected from the group consisting of S, T, and K.

2. A method for treating and/or limiting scars selected from the group consisting of keloids and hypertrophic scars comprising administering to an individual in need thereof an amount effective to treat and/or limit scars selected from the group consisting of keloids and hypertrophic scars of a polypeptide comprising a sequence according to general formula I:
X1-A(X2)APLP-X3
wherein X1 is 0-14 amino acids of the sequence of heat shock protein 20 between residues 1 and 14 of SEQ ID NO: 298;
X2 is selected from the group consisting of S, T, Y, D, E, hydroxylysine, hydroxyproline, phosphoserine analogs, and phosphotyrosine analogs; and
X3 is selected from the group consisting of (a) 0-140 amino acids of residues 21 and 160 of SEQ ID NO:298; and (b) 0, 1, 2, or 3 amino acids of a sequence of genus Z1-Z2-Z3, wherein Z1 is selected from the group consisting of G and D;
Z2 is selected from the group consisting of L and K; and
Z3 is selected from the group consisting of S, T, and K.

3. The method of claim 1 or 2 wherein X1 is WLRR (SEQ ID NO: 1).

4. The method of claim 1 or 2 wherein X3 is GLK.

5. The method of claim 1 or 2 wherein X1 is 0-14 amino acids of the sequence of heat shock protein 20 between residues 1 and 14 of SEQ ID NO: 298.

6. The method of claim 1 or 2 wherein the polypeptide comprises WLRRASAPLPGLK (SEQ ID NO: 300), wherein the S residue is phosphorylated.

7. The method of claim 1 or 2, wherein the polypeptide further comprises a covalently bound transduction domain.

8. The method of claim 7, wherein the transduction domain comprises a polypeptide selected from the group consisting of YARAAARQARA (SEQ ID NO: 281) and YGRKKRRQRRR (SEQ ID NO: 299).

9. The method of claim 2 wherein the method is used to limit scars selected from the group consisting of keloids and hypertrophic scars, and wherein the individual in need thereof is of Asian or African descent.

10. The method of claim 1 or 2 wherein the method is used to treat or limit fibrotic disorders, and wherein the individual in need thereof is suffering from or at risk of one or more of diabetic nephropathy, glomerulosclerosis, IgA nephropathy, diabetic retinopathy, macular degeneration, cirrhosis, biliary atresia, congestive heart failure, scleroderma, and abdominal adhesions.

11. The method of claim 1 or 2, wherein the individual in need thereof has an elevated level of one or more of the following biomarkers in a target tissue:
TGFβ1 expression;
TGFβ2 expression;
CTGF expression;
Phosphorylated cofilin;
Phosphorylated HSP27; and
α-smooth muscle actin expression.

12. The method of claim 1 or 2, further comprising monitoring effectiveness of the method by determining the levels after initiation of the method of one or more of the following biomarkers in a target tissue:
TGFβ1 expression;
TGFβ2 expression;
CTGF expression;
Phosphorylated cofilin;
Phosphorylated HSP27; and
α-smooth muscle actin expression.


SEQUENCE LISTING (321 sequences)

SEQ ID NO: 1 - protein, 4 aa, Artificial
  Synthetic
WLRR

SEQ ID NO: 2 - protein, 6 aa, Artificial
  Synthetic
AXAPLP

SEQ ID NO: 3 - protein, 6 aa, Artificial
  Synthetic
ASAPLP

SEQ ID NO: 4 - protein, 6 aa, Artificial
  Synthetic
ATAPLP

SEQ ID NO: 5 - protein, 7 aa, Artificial
  Synthetic
RASAPLP

SEQ ID NO: 6 - protein, 7 aa, Artificial
  Synthetic
RATAPLP

SEQ ID NO: 7 - protein, 6 aa, Artificial
  Synthetic
AYAPLP

SEQ ID NO: 8 - protein, 7 aa, Artificial
  Synthetic
RAYAPLP

SEQ ID NO: 9 - protein, 8 aa, Artificial
  Synthetic
RRASAPLP

SEQ ID NO: 10 - protein, 9 aa, Artificial
  Synthetic
LRRASAPLP

SEQ ID NO: 11 - protein, 10 aa, Artificial
  Synthetic
WLRRASAPLP

SEQ ID NO: 12 - protein, 8 aa, Artificial
  Synthetic
RRATAPLP

SEQ ID NO: 13 - protein, 9 aa, Artificial
  Synthetic
LRRATAPLP

SEQ ID NO: 14 - protein, 10 aa, Artificial
  Synthetic
WLRRATAPLP

SEQ ID NO: 15 - protein, 8 aa, Artificial
  Synthetic
RRAYAPLP

SEQ ID NO: 16 - protein, 9 aa, Artificial
  Synthetic
LRRAYAPLP

SEQ ID NO: 17 - protein, 10 aa, Artificial
  Synthetic
WLRRAYAPLP

SEQ ID NO: 18 - protein, 9 aa, Artificial
  Synthetic
RRASAPLPG

SEQ ID NO: 19 - protein, 9 aa, Artificial
  Synthetic
RRASAPLPD

SEQ ID NO: 20 - protein, 10 aa, Artificial
  Synthetic
RRASAPLPGL

SEQ ID NO: 21 - protein, 10 aa, Artificial
  Synthetic
RRASAPLPGK

SEQ ID NO: 22 - protein, 10 aa, Artificial
  Synthetic
RRASAPLPDL

SEQ ID NO: 23 - protein, 10 aa, Artificial
  Synthetic
RRASAPLPDK

SEQ ID NO: 24 - protein, 11 aa, Artificial
  Synthetic
RRASAPLPGLS

SEQ ID NO: 25 - protein, 11 aa, Artificial
  Synthetic
RRASAPLPGLT

SEQ ID NO: 26 - protein, 11 aa, Artificial
  Synthetic
RRASAPLPGKS

SEQ ID NO: 27 - protein, 11 aa, Artificial
  Synthetic
RRASAPLPGKT

SEQ ID NO: 28 - protein, 11 aa, Artificial
  Synthetic
RRASAPLPDLS

SEQ ID NO: 29 - protein, 11 aa, Artificial
  Synthetic
RRASAPLPDLT

SEQ ID NO: 30 - protein, 11 aa, Artificial
  Synthetic
RRASAPLPDKS

SEQ ID NO: 31 - protein, 11 aa, Artificial
  Synthetic
RRASAPLPDKT

SEQ ID NO: 32 - protein, 10 aa, Artificial
  Synthetic
LRRASAPLPG

SEQ ID NO: 33 - protein, 10 aa, Artificial
  Synthetic
LRRASAPLPD

SEQ ID NO: 34 - protein, 11 aa, Artificial
  Synthetic
LRRASAPLPGL

SEQ ID NO: 35 - protein, 11 aa, Artificial
  Synthetic
LRRASAPLPGK

SEQ ID NO: 36 - protein, 11 aa, Artificial
  Synthetic
LRRASAPLPDL

SEQ ID NO: 37 - protein, 11 aa, Artificial
  Synthetic
LRRASAPLPDK

SEQ ID NO: 38 - protein, 12 aa, Artificial
  Synthetic
LRRASAPLPGLS

SEQ ID NO: 39 - protein, 12 aa, Artificial
  Synthetic
LRRASAPLPGLT

SEQ ID NO: 40 - protein, 12 aa, Artificial
  Synthetic
LRRASAPLPGKS

SEQ ID NO: 41 - protein, 12 aa, Artificial
  Synthetic
LRRASAPLPGKT

SEQ ID NO: 42 - protein, 12 aa, Artificial
  Synthetic
LRRASAPLPDLS

SEQ ID NO: 43 - protein, 12 aa, Artificial
  Synthetic
LRRASAPLPDLT

SEQ ID NO: 44 - protein, 12 aa, Artificial
  Synthetic
LRRASAPLPDKS

SEQ ID NO: 45 - protein, 12 aa, Artificial
  Synthetic
LRRASAPLPDKT

SEQ ID NO: 46 - protein, 11 aa, Artificial
  Synthetic
WLRRASAPLPG

SEQ ID NO: 47 - protein, 11 aa, Artificial
  Synthetic
WLRRASAPLPD

SEQ ID NO: 48 - protein, 12 aa, Artificial
  Synthetic
WLRRASAPLPGL

SEQ ID NO: 49 - protein, 12 aa, Artificial
  Synthetic
WLRRASAPLPGK

SEQ ID NO: 50 - protein, 12 aa, Artificial
  Synthetic
WLRRASAPLPDL

SEQ ID NO: 51 - protein, 12 aa, Artificial
  Synthetic
WLRRASAPLPDK

SEQ ID NO: 52 - protein, 13 aa, Artificial
  Synthetic
WLRRASAPLPGLS

SEQ ID NO: 53 - protein, 13 aa, Artificial
  Synthetic
WLRRASAPLPGLT

SEQ ID NO: 54 - protein, 13 aa, Artificial
  Synthetic
WLRRASAPLPGKS

SEQ ID NO: 55 - protein, 13 aa, Artificial
  Synthetic
WLRRASAPLPGKT

SEQ ID NO: 56 - protein, 13 aa, Artificial
  Synthetic
WLRRASAPLPDLS

SEQ ID NO: 57 - protein, 13 aa, Artificial
  Synthetic
WLRRASAPLPDLT

SEQ ID NO: 58 - protein, 13 aa, Artificial
  Synthetic
WLRRASAPLPDKS

SEQ ID NO: 59 - protein, 13 aa, Artificial
  Synthetic
WLRRASAPLPDKT

SEQ ID NO: 60 - protein, 9 aa, Artificial
  Synthetic
RRATAPLPG

SEQ ID NO: 61 - protein, 9 aa, Artificial
  Synthetic
RRATAPLPD

SEQ ID NO: 62 - protein, 10 aa, Artificial
  Synthetic
RRATAPLPGL

SEQ ID NO: 63 - protein, 10 aa, Artificial
  Synthetic
RRATAPLPGK

SEQ ID NO: 64 - protein, 10 aa, Artificial
  Synthetic
RRATAPLPDL

SEQ ID NO: 65 - protein, 10 aa, Artificial
  Synthetic
RRATAPLPDK

SEQ ID NO: 66 - protein, 11 aa, Artificial
  Synthetic
RRATAPLPGLS

SEQ ID NO: 67 - protein, 11 aa, Artificial
  Synthetic
RRATAPLPGLT

SEQ ID NO: 68 - protein, 11 aa, Artificial
  Synthetic
RRATAPLPGKS

SEQ ID NO: 69 - protein, 11 aa, Artificial
  Synthetic
RRATAPLPGKT

SEQ ID NO: 70 - protein, 11 aa, Artificial
  Synthetic
RRATAPLPDLS

SEQ ID NO: 71 - protein, 11 aa, Artificial
  Synthetic
RRATAPLPDLT

SEQ ID NO: 72 - protein, 11 aa, Artificial
  Synthetic
RRATAPLPDKS

SEQ ID NO: 73 - protein, 11 aa, Artificial
  Synthetic
RRATAPLPDKT

SEQ ID NO: 74 - protein, 10 aa, Artificial
  Synthetic
LRRATAPLPG

SEQ ID NO: 75 - protein, 10 aa, Artificial
  Synthetic
LRRATAPLPD

SEQ ID NO: 76 - protein, 11 aa, Artificial
  Synthetic
LRRATAPLPGL

SEQ ID NO: 77 - protein, 11 aa, Artificial
  Synthetic
LRRATAPLPGK

SEQ ID NO: 78 - protein, 11 aa, Artificial
  Synthetic
LRRATAPLPDL

SEQ ID NO: 79 - protein, 11 aa, Artificial
  Synthetic
LRRATAPLPDK

SEQ ID NO: 80 - protein, 12 aa, Artificial
  Synthetic
LRRATAPLPGLS

SEQ ID NO: 81 - protein, 12 aa, Artificial
  Synthetic
LRRATAPLPGLT

SEQ ID NO: 82 - protein, 12 aa, Artificial
  Synthetic
LRRATAPLPGKS

SEQ ID NO: 83 - protein, 12 aa, Artificial
  Synthetic
LRRATAPLPGKT

SEQ ID NO: 84 - protein, 12 aa, Artificial
  Synthetic
LRRATAPLPDLS

SEQ ID NO: 85 - protein, 12 aa, Artificial
  Synthetic
LRRATAPLPDLT

SEQ ID NO: 86 - protein, 12 aa, Artificial
  Synthetic
LRRATAPLPDKS

SEQ ID NO: 87 - protein, 12 aa, Artificial
  Synthetic
LRRATAPLPDKT

SEQ ID NO: 88 - protein, 11 aa, Artificial
  Synthetic
WLRRATAPLPG

SEQ ID NO: 89 - protein, 11 aa, Artificial
  Synthetic
WLRRATAPLPD

SEQ ID NO: 90 - protein, 12 aa, Artificial
  Synthetic
WLRRATAPLPGL

SEQ ID NO: 91 - protein, 12 aa, Artificial
  Synthetic
WLRRATAPLPGK

SEQ ID NO: 92 - protein, 12 aa, Artificial
  Synthetic
WLRRATAPLPDL

SEQ ID NO: 93 - protein, 12 aa, Artificial
  Synthetic
WLRRATAPLPDK

SEQ ID NO: 94 - protein, 13 aa, Artificial
  Synthetic
WLRRATAPLPGLS

SEQ ID NO: 95 - protein, 13 aa, Artificial
  Synthetic
WLRRATAPLPGLT

SEQ ID NO: 96 - protein, 13 aa, Artificial
  Synthetic
WLRRATAPLPGKS

SEQ ID NO: 97 - protein, 13 aa, Artificial
  Synthetic
WLRRATAPLPGKT

SEQ ID NO: 98 - protein, 13 aa, Artificial
  Synthetic
WLRRATAPLPDLS

SEQ ID NO: 99 - protein, 13 aa, Artificial
  Synthetic
WLRRATAPLPDLT

SEQ ID NO: 100 - protein, 13 aa, Artificial
  Synthetic
WLRRATAPLPDKS

SEQ ID NO: 101 - protein, 13 aa, Artificial
  Synthetic
WLRRATAPLPDKT

SEQ ID NO: 102 - protein, 9 aa, Artificial
  Synthetic
RRAYAPLPG

SEQ ID NO: 103 - protein, 9 aa, Artificial
  Synthetic
RRAYAPLPD

SEQ ID NO: 104 - protein, 10 aa, Artificial
  Synthetic
RRAYAPLPGL

SEQ ID NO: 105 - protein, 10 aa, Artificial
  Synthetic
RRAYAPLPGK

SEQ ID NO: 106 - protein, 10 aa, Artificial
  Synthetic
RRAYAPLPDL

SEQ ID NO: 107 - protein, 10 aa, Artificial
  Synthetic
RRAYAPLPDK

SEQ ID NO: 108 - protein, 11 aa, Artificial
  Synthetic
RRAYAPLPGLS

SEQ ID NO: 109 - protein, 11 aa, Artificial
  Synthetic
RRAYAPLPGLT

SEQ ID NO: 110 - protein, 11 aa, Artificial
  Synthetic
RRAYAPLPGKS

SEQ ID NO: 111 - protein, 11 aa, Artificial
  Synthetic
RRAYAPLPGKT

SEQ ID NO: 112 - protein, 11 aa, Artificial
  Synthetic
RRAYAPLPDLS

SEQ ID NO: 113 - protein, 11 aa, Artificial
  Synthetic
RRAYAPLPDLT

SEQ ID NO: 114 - protein, 11 aa, Artificial
  Synthetic
RRAYAPLPDKS

SEQ ID NO: 115 - protein, 11 aa, Artificial
  Synthetic
RRAYAPLPDKT

SEQ ID NO: 116 - protein, 10 aa, Artificial
  Synthetic
LRRAYAPLPG

SEQ ID NO: 117 - protein, 10 aa, Artificial
  Synthetic
LRRAYAPLPD

SEQ ID NO: 118 - protein, 11 aa, Artificial
  Synthetic
LRRAYAPLPGL

SEQ ID NO: 119 - protein, 11 aa, Artificial
  Synthetic
LRRAYAPLPGK

SEQ ID NO: 120 - protein, 11 aa, Artificial
  Synthetic
LRRAYAPLPDL

SEQ ID NO: 121 - protein, 11 aa, Artificial
  Synthetic
LRRAYAPLPDK

SEQ ID NO: 122 - protein, 12 aa, Artificial
  Synthetic
LRRAYAPLPGLS

SEQ ID NO: 123 - protein, 12 aa, Artificial
  Synthetic
LRRAYAPLPGLT

SEQ ID NO: 124 - protein, 12 aa, Artificial
  Synthetic
LRRAYAPLPGKS

SEQ ID NO: 125 - protein, 12 aa, Artificial
  Synthetic
LRRAYAPLPGKT

SEQ ID NO: 126 - protein, 12 aa, Artificial
  Synthetic
LRRAYAPLPDLS

SEQ ID NO: 127 - protein, 12 aa, Artificial
  Synthetic
LRRAYAPLPDLT

SEQ ID NO: 128 - protein, 12 aa, Artificial
  Synthetic
LRRAYAPLPDKS

SEQ ID NO: 129 - protein, 12 aa, Artificial
  Synthetic
LRRAYAPLPDKT

SEQ ID NO: 130 - protein, 11 aa, Artificial
  Synthetic
WLRRAYAPLPG

SEQ ID NO: 131 - protein, 11 aa, Artificial
  Synthetic
WLRRAYAPLPD

SEQ ID NO: 132 - protein, 12 aa, Artificial
  Synthetic
WLRRAYAPLPGL

SEQ ID NO: 133 - protein, 12 aa, Artificial
  Synthetic
WLRRAYAPLPGK

SEQ ID NO: 134 - protein, 12 aa, Artificial
  Synthetic
WLRRAYAPLPDL

SEQ ID NO: 135 - protein, 12 aa, Artificial
  Synthetic
WLRRAYAPLPDK

SEQ ID NO: 136 - protein, 13 aa, Artificial
  Synthetic
WLRRAYAPLPGLS

SEQ ID NO: 137 - protein, 13 aa, Artificial
  Synthetic
WLRRAYAPLPGLT

SEQ ID NO: 138 - protein, 13 aa, Artificial
  Synthetic
WLRRAYAPLPGKS

SEQ ID NO: 139 - protein, 13 aa, Artificial
  Synthetic
WLRRAYAPLPGKT

SEQ ID NO: 140 - protein, 13 aa, Artificial
  Synthetic
WLRRAYAPLPDLS

SEQ ID NO: 141 - protein, 13 aa, Artificial
  Synthetic
WLRRAYAPLPDLT

SEQ ID NO: 142 - protein, 13 aa, Artificial
  Synthetic
WLRRAYAPLPDKS

SEQ ID NO: 143 - protein, 13 aa, Artificial
  Synthetic
WLRRAYAPLPDKT

SEQ ID NO: 144 - protein, 9 aa, Artificial
  Synthetic
XRRASAPLP

SEQ ID NO: 145 - protein, 10 aa, Artificial
  Synthetic
XLRRASAPLP

SEQ ID NO: 146 - protein, 11 aa, Artificial
  Synthetic
XWLRRASAPLP

SEQ ID NO: 147 - protein, 9 aa, Artificial
  Synthetic
XRRATAPLP

SEQ ID NO: 148 - protein, 10 aa, Artificial
  Synthetic
XLRRATAPLP

SEQ ID NO: 149 - protein, 11 aa, Artificial
  Synthetic
XWLRRATAPLP

SEQ ID NO: 150 - protein, 9 aa, Artificial
  Synthetic
XRRAYAPLP

SEQ ID NO: 151 - protein, 10 aa, Artificial
  Synthetic
XLRRAYAPLP

SEQ ID NO: 152 - protein, 11 aa, Artificial
  Synthetic
XWLRRAYAPLP

SEQ ID NO: 153 - protein, 10 aa, Artificial
  Synthetic
XRRASAPLPG

SEQ ID NO: 154 - protein, 10 aa, Artificial
  Synthetic
XRRASAPLPD

SEQ ID NO: 155 - protein, 11 aa, Artificial
  Synthetic
XRRASAPLPGL

SEQ ID NO: 156 - protein, 11 aa, Artificial
  Synthetic
XRRASAPLPGK

SEQ ID NO: 157 - protein, 11 aa, Artificial
  Synthetic
XRRASAPLPDL

SEQ ID NO: 158 - protein, 11 aa, Artificial
  Synthetic
XRRASAPLPDK

SEQ ID NO: 159 - protein, 12 aa, Artificial
  Synthetic
XRRASAPLPGLS

SEQ ID NO: 160 - protein, 12 aa, Artificial
  Synthetic
XRRASAPLPGLT

SEQ ID NO: 161 - protein, 12 aa, Artificial
  Synthetic
XRRASAPLPGKS

SEQ ID NO: 162 - protein, 12 aa, Artificial
  Synthetic
XRRASAPLPGKT

SEQ ID NO: 163 - protein, 12 aa, Artificial
  Synthetic
XRRASAPLPDLS

SEQ ID NO: 164 - protein, 12 aa, Artificial
  Synthetic
XRRASAPLPDLT

SEQ ID NO: 165 - protein, 12 aa, Artificial
  Synthetic
XRRASAPLPDKS

SEQ ID NO: 166 - protein, 12 aa, Artificial
  Synthetic
XRRASAPLPDKT

SEQ ID NO: 167 - protein, 11 aa, Artificial
  Synthetic
XLRRASAPLPG

SEQ ID NO: 168 - protein, 11 aa, Artificial
  Synthetic
XLRRASAPLPD

SEQ ID NO: 169 - protein, 12 aa, Artificial
  Synthetic
XLRRASAPLPGL

SEQ ID NO: 170 - protein, 12 aa, Artificial
  Synthetic
XLRRASAPLPGK

SEQ ID NO: 171 - protein, 12 aa, Artificial
  Synthetic
XLRRASAPLPDL

SEQ ID NO: 172 - protein, 12 aa, Artificial
  Synthetic
XLRRASAPLPDK

SEQ ID NO: 173 - protein, 13 aa, Artificial
  Synthetic
XLRRASAPLPGLS

SEQ ID NO: 174 - protein, 13 aa, Artificial
  Synthetic
XLRRASAPLPGLT

SEQ ID NO: 175 - protein, 13 aa, Artificial
  Synthetic
XLRRASAPLPGKS

SEQ ID NO: 176 - protein, 13 aa, Artificial
  Synthetic
XLRRASAPLPGKT

SEQ ID NO: 177 - protein, 13 aa, Artificial
  Synthetic
XLRRASAPLPDLS

SEQ ID NO: 178 - protein, 13 aa, Artificial
  Synthetic
XLRRASAPLPDLT

SEQ ID NO: 179 - protein, 13 aa, Artificial
  Synthetic
XLRRASAPLPDKS

SEQ ID NO: 180 - protein, 13 aa, Artificial
  Synthetic
XLRRASAPLPDKT

SEQ ID NO: 181 - protein, 12 aa, Artificial
  Synthetic
XWLRRASAPLPG

SEQ ID NO: 182 - protein, 12 aa, Artificial
  Synthetic
XWLRRASAPLPD

SEQ ID NO: 183 - protein, 13 aa, Artificial
  Synthetic
XWLRRASAPLPGL

SEQ ID NO: 184 - protein, 13 aa, Artificial
  Synthetic
XWLRRASAPLPGK

SEQ ID NO: 185 - protein, 13 aa, Artificial
  Synthetic
XWLRRASAPLPDL

SEQ ID NO: 186 - protein, 13 aa, Artificial
  Synthetic
XWLRRASAPLPDK

SEQ ID NO: 187 - protein, 14 aa, Artificial
  Synthetic
XWLRRASAPLPGLS

SEQ ID NO: 188 - protein, 14 aa, Artificial
  Synthetic
XWLRRASAPLPGLT

SEQ ID NO: 189 - protein, 14 aa, Artificial
  Synthetic
XWLRRASAPLPGKS

SEQ ID NO: 190 - protein, 14 aa, Artificial
  Synthetic
XWLRRASAPLPGKT

SEQ ID NO: 191 - protein, 14 aa, Artificial
  Synthetic
XWLRRASAPLPDLS

SEQ ID NO: 192 - protein, 14 aa, Artificial
  Synthetic
XWLRRASAPLPDLT

SEQ ID NO: 193 - protein, 14 aa, Artificial
  Synthetic
XWLRRASAPLPDKS

SEQ ID NO: 194 - protein, 14 aa, Artificial
  Synthetic
XWLRRASAPLPDKT

SEQ ID NO: 195 - protein, 10 aa, Artificial
  Synthetic
XRRATAPLPG

SEQ ID NO: 196 - protein, 10 aa, Artificial
  Synthetic
XRRATAPLPD

SEQ ID NO: 197 - protein, 11 aa, Artificial
  Synthetic
XRRATAPLPGL

SEQ ID NO: 198 - protein, 11 aa, Artificial
  Synthetic
XRRATAPLPGK

SEQ ID NO: 199 - protein, 11 aa, Artificial
  Synthetic
XRRATAPLPDL

SEQ ID NO: 200 - protein, 11 aa, Artificial
  Synthetic
XRRATAPLPDK

SEQ ID NO: 201 - protein, 12 aa, Artificial
  Synthetic
XRRATAPLPGLS

SEQ ID NO: 202 - protein, 12 aa, Artificial
  Synthetic
XRRATAPLPGLT

SEQ ID NO: 203 - protein, 12 aa, Artificial
  Synthetic
XRRATAPLPGKS

SEQ ID NO: 204 - protein, 12 aa, Artificial
  Synthetic
XRRATAPLPGKT

SEQ ID NO: 205 - protein, 12 aa, Artificial
  Synthetic
XRRATAPLPDLS

SEQ ID NO: 206 - protein, 12 aa, Artificial
  Synthetic
XRRATAPLPDLT

SEQ ID NO: 207 - protein, 12 aa, Artificial
  Synthetic
XRRATAPLPDKS

SEQ ID NO: 208 - protein, 12 aa, Artificial
  Synthetic
XRRATAPLPDKT

SEQ ID NO: 209 - protein, 11 aa, Artificial
  Synthetic
XLRRATAPLPG

SEQ ID NO: 210 - protein, 11 aa, Artificial
  Synthetic
XLRRATAPLPD

SEQ ID NO: 211 - protein, 12 aa, Artificial
  Synthetic
XLRRATAPLPGL

SEQ ID NO: 212 - protein, 12 aa, Artificial
  Synthetic
XLRRATAPLPGK

SEQ ID NO: 213 - protein, 12 aa, Artificial
  Synthetic
XLRRATAPLPDL

SEQ ID NO: 214 - protein, 12 aa, Artificial
  Synthetic
XLRRATAPLPDK

SEQ ID NO: 215 - protein, 13 aa, Artificial
  Synthetic
XLRRATAPLPGLS

SEQ ID NO: 216 - protein, 13 aa, Artificial
  Synthetic
XLRRATAPLPGLT

SEQ ID NO: 217 - protein, 13 aa, Artificial
  Synthetic
XLRRATAPLPGKS

SEQ ID NO: 218 - protein, 13 aa, Artificial
  Synthetic
XLRRATAPLPGKT

SEQ ID NO: 219 - protein, 13 aa, Artificial
  Synthetic
XLRRATAPLPDLS

SEQ ID NO: 220 - protein, 13 aa, Artificial
  Synthetic
XLRRATAPLPDLT

SEQ ID NO: 221 - protein, 13 aa, Artificial
  Synthetic
XLRRATAPLPDKS

SEQ ID NO: 222 - protein, 13 aa, Artificial
  Synthetic
XLRRATAPLPDKT

SEQ ID NO: 223 - protein, 12 aa, Artificial
  Synthetic
XWLRRATAPLPG

SEQ ID NO: 224 - protein, 12 aa, Artificial
  Synthetic
XWLRRATAPLPD

SEQ ID NO: 225 - protein, 13 aa, Artificial
  Synthetic
XWLRRATAPLPGL

SEQ ID NO: 226 - protein, 13 aa, Artificial
  Synthetic
XWLRRATAPLPGK

SEQ ID NO: 227 - protein, 13 aa, Artificial
  Synthetic
XWLRRATAPLPDL

SEQ ID NO: 228 - protein, 13 aa, Artificial
  Synthetic
XWLRRATAPLPDK

SEQ ID NO: 229 - protein, 14 aa, Artificial
  Synthetic
XWLRRATAPLPGLS

SEQ ID NO: 230 - protein, 14 aa, Artificial
  Synthetic
XWLRRATAPLPGLT

SEQ ID NO: 231 - protein, 14 aa, Artificial
  Synthetic
XWLRRATAPLPGKS

SEQ ID NO: 232 - protein, 14 aa, Artificial
  Synthetic
XWLRRATAPLPGKT

SEQ ID NO: 233 - protein, 14 aa, Artificial
  Synthetic
XWLRRATAPLPDLS

SEQ ID NO: 234 - protein, 14 aa, Artificial
  Synthetic
XWLRRATAPLPDLT

SEQ ID NO: 235 - protein, 14 aa, Artificial
  Synthetic
XWLRRATAPLPDKS

SEQ ID NO: 236 - protein, 14 aa, Artificial
  Synthetic
XWLRRATAPLPDKT

SEQ ID NO: 237 - protein, 10 aa, Artificial
  Synthetic
XRRAYAPLPG

SEQ ID NO: 238 - protein, 10 aa, Artificial
  Synthetic
XRRAYAPLPD

SEQ ID NO: 239 - protein, 11 aa, Artificial
  Synthetic
XRRAYAPLPGL

SEQ ID NO: 240 - protein, 11 aa, Artificial
  Synthetic
XRRAYAPLPGK

SEQ ID NO: 241 - protein, 11 aa, Artificial
  Synthetic
XRRAYAPLPDL

SEQ ID NO: 242 - protein, 11 aa, Artificial
  Synthetic
XRRAYAPLPDK

SEQ ID NO: 243 - protein, 12 aa, Artificial
  Synthetic
XRRAYAPLPGLS

SEQ ID NO: 244 - protein, 12 aa, Artificial
  Synthetic
XRRAYAPLPGLT

SEQ ID NO: 245 - protein, 12 aa, Artificial
  Synthetic
XRRAYAPLPGKS

SEQ ID NO: 246 - protein, 12 aa, Artificial
  Synthetic
XRRAYAPLPGKT

SEQ ID NO: 247 - protein, 12 aa, Artificial
  Synthetic
XRRAYAPLPDLS

SEQ ID NO: 248 - protein, 12 aa, Artificial
  Synthetic
XRRAYAPLPDLT

SEQ ID NO: 249 - protein, 12 aa, Artificial
  Synthetic
XRRAYAPLPDKS

SEQ ID NO: 250 - protein, 12 aa, Artificial
  Synthetic
XRRAYAPLPDKT

SEQ ID NO: 251 - protein, 11 aa, Artificial
  Synthetic
XLRRAYAPLPG

SEQ ID NO: 252 - protein, 11 aa, Artificial
  Synthetic
XLRRAYAPLPD

SEQ ID NO: 253 - protein, 12 aa, Artificial
  Synthetic
XLRRAYAPLPGL

SEQ ID NO: 254 - protein, 12 aa, Artificial
  Synthetic
XLRRAYAPLPGK

SEQ ID NO: 255 - protein, 12 aa, Artificial
  Synthetic
XLRRAYAPLPDL

SEQ ID NO: 256 - protein, 12 aa, Artificial
  Synthetic
XLRRAYAPLPDK

SEQ ID NO: 257 - protein, 13 aa, Artificial
  Synthetic
XLRRAYAPLPGLS

SEQ ID NO: 258 - protein, 13 aa, Artificial
  Synthetic
XLRRAYAPLPGLT

SEQ ID NO: 259 - protein, 13 aa, Artificial
  Synthetic
XLRRAYAPLPGKS

SEQ ID NO: 260 - protein, 13 aa, Artificial
  Synthetic
XLRRAYAPLPGKT

SEQ ID NO: 261 - protein, 13 aa, Artificial
  Synthetic
XLRRAYAPLPDLS

SEQ ID NO: 262 - protein, 13 aa, Artificial
  Synthetic
XLRRAYAPLPDLT

SEQ ID NO: 263 - protein, 13 aa, Artificial
  Synthetic
XLRRAYAPLPDKS

SEQ ID NO: 264 - protein, 13 aa, Artificial
  Synthetic
XLRRAYAPLPDKT

SEQ ID NO: 265 - protein, 12 aa, Artificial
  Synthetic
XWLRRAYAPLPG

SEQ ID NO: 266 - protein, 12 aa, Artificial
  Synthetic
XWLRRAYAPLPD

SEQ ID NO: 267 - protein, 13 aa, Artificial
  Synthetic
XWLRRAYAPLPGL

SEQ ID NO: 268 - protein, 13 aa, Artificial
  Synthetic
XWLRRAYAPLPGK

SEQ ID NO: 269 - protein, 13 aa, Artificial
  Synthetic
XWLRRAYAPLPDL

SEQ ID NO: 270 - protein, 13 aa, Artificial
  Synthetic
XWLRRAYAPLPDK

SEQ ID NO: 271 - protein, 14 aa, Artificial
  Synthetic
XWLRRAYAPLPGLS

SEQ ID NO: 272 - protein, 14 aa, Artificial
  Synthetic
XWLRRAYAPLPGLT

SEQ ID NO: 273 - protein, 14 aa, Artificial
  Synthetic
XWLRRAYAPLPGKS

SEQ ID NO: 274 - protein, 14 aa, Artificial
  Synthetic
XWLRRAYAPLPGKT

SEQ ID NO: 275 - protein, 14 aa, Artificial
  Synthetic
XWLRRAYAPLPDLS

SEQ ID NO: 276 - protein, 14 aa, Artificial
  Synthetic
XWLRRAYAPLPDLT

SEQ ID NO: 277 - protein, 14 aa, Artificial
  Synthetic
XWLRRAYAPLPDKS

SEQ ID NO: 278 - protein, 14 aa, Artificial
  Synthetic
XWLRRAYAPLPDKT

SEQ ID NO: 279 - protein, 9 aa, Artificial
  Synthetic
RRRRRRRRR

SEQ ID NO: 280 - protein, 13 aa, Artificial
  Synthetic
GRKKRRQRRRPPQ

SEQ ID NO: 281 - protein, 11 aa, Artificial
  Synthetic
YARAAARQARA

SEQ ID NO: 282 - protein, 34 aa, Artificial
  Synthetic
DAATATRGRSAASRPTERPRAPARSASRPRRPVE

SEQ ID NO: 283 - protein, 27 aa, Artificial
  Synthetic
GWTLNSAGYLLGLINLKALAALAKKIL

SEQ ID NO: 284 - protein, 12 aa, Artificial
  Synthetic
PLSSIFSRIGDP

SEQ ID NO: 285 - protein, 16 aa, Artificial
  Synthetic
AAVALLPAVLLALLAP

SEQ ID NO: 286 - protein, 12 aa, Artificial
  Synthetic
AAVLLPVLLAAP

SEQ ID NO: 287 - protein, 15 aa, Artificial
  Synthetic
VTVLALGALAGVGVG

SEQ ID NO: 288 - protein, 21 aa, Artificial
  Synthetic
GALFLGWLGAAGSTMGAWSQP

SEQ ID NO: 289 - protein, 27 aa, Artificial
  Synthetic
GWTLNSAGYLLGLINLKALAALAKKIL

SEQ ID NO: 290 - protein, 18 aa, Artificial
  Synthetic
KLALKLALKALKAALKLA

SEQ ID NO: 291 - protein, 21 aa, Artificial
  Synthetic
KETWWETWWTEWSQPKKKRKV

SEQ ID NO: 292 - protein, 16 aa, Artificial
  Synthetic
KAFAKLAARLYRKAGC

SEQ ID NO: 293 - protein, 16 aa, Artificial
  Synthetic
KAFAKLAARLYRAAGC

SEQ ID NO: 294 - protein, 16 aa, Artificial
  Synthetic
AAFAKLAARLYRKAGC

SEQ ID NO: 295 - protein, 16 aa, Artificial
  Synthetic
KAFAALAARLYRKAGC

SEQ ID NO: 296 - protein, 16 aa, Artificial
  Synthetic
KAFAKLAAQLYRKAGC

SEQ ID NO: 297 - protein, 15 aa, Artificial
  Synthetic
GGGGYGRKKRRQRRR

SEQ ID NO: 298 - protein, 160 aa, Artificial
  Synthetic
MEIPVPVQPSWLRRASAPLPGLSAPGRLFDQRFGEGLLEAELAALCPTTLAPYYLRAPSVALPVAQVPTDPGHFSVLLDVKHFSPEEIAVKVVGEHVEVHARHEERPDEHGFVAREFHRRYRLPPGVDPAAVTSALSPEGVLSIQAAPASAQAPPPAAAK

SEQ ID NO: 299 - protein, 11 aa, Artificial
  Synthetic
YGRKKRRQRRR

SEQ ID NO: 300 - protein, 13 aa, Artificial
  Synthetic
WLRRASAPLPGLK

SEQ ID NO: 301 - protein, 23 aa, Artificial
  Synthetic
YGRKKRRQRRRWLRRASAPLPGL

SEQ ID NO: 302 - protein, 160 aa, Artificial
  Synthetic
MEIPVPVQPSWLRRAXAPLPGLSAPGRLFDQRFGEGLLEAELAALCPTTLAPYYLRAPSVALPVAQVPTDPGHFSVLLDVKHFSPEEIAVKVVGEHVEVHARHEERPDEHGFVAREFHRRYRLPPGVDPAAVTSALSPEGVLSIQAAPASAQAPPPAAAK

SEQ ID NO: 303 - protein, 5 aa, Artificial
  Synthetic
SWLRR

SEQ ID NO: 304 - protein, 3 aa, Artificial
  Synthetic
LRR

SEQ ID NO: 305 - protein, 6 aa, Artificial
  Synthetic
PSWLRR

SEQ ID NO: 306 - protein, 7 aa, Artificial
  Synthetic
NPSWLRR

SEQ ID NO: 307 - protein, 8 aa, Artificial
  Synthetic
VNPSWLRR

SEQ ID NO: 308 - protein, 9 aa, Artificial
  Synthetic
PVNPSWLRR

SEQ ID NO: 309 - protein, 10 aa, Artificial
  Synthetic
VPVNPSWLRR

SEQ ID NO: 310 - protein, 11 aa, Artificial
  Synthetic
PVPVNPSWLRR

SEQ ID NO: 311 - protein, 13 aa, Artificial
  Synthetic
IPVPPVNPSWLRR

SEQ ID NO: 312 - protein, 14 aa, Artificial
  Synthetic
EIPVPPVNPSWLRR

SEQ ID NO: 313 - protein, 15 aa, Artificial
  Synthetic
MEIPVPPVNPSWLRR

SEQ ID NO: 314 - protein, 5 aa, Artificial
  Synthetic
GLSAP

SEQ ID NO: 315 - protein, 23 aa, Artificial
  Synthetic
YARAAARQARAWLRRASAPLPGL

SEQ ID NO: 316 - protein, 23 aa, Artificial
  Synthetic
MEIPVPVQPSWLRRAXAPLPXXX

SEQ ID NO: 317 - protein, 35 aa, Artificial
  Synthetic
YXRXXXRQXRXWLRRASAPLPGLKYXRXXXRQXRX

SEQ ID NO: 318 - protein, 182 aa, Artificial
  Synthetic
YXRXXXRQXRXMEIPVPVQPSWLRRAXAPLPGLSAPGRLFDQRFGEGLLEAELAALCPTTLAPYYLRAPSVALPVAQVPTDPGHFSVLLDVKHFSPEEIAVKVVGEHVEVHARHEERPDEHGFVAREFHRRYRLPPGVDPAAVTSALSPEGVLSIQAAPASAQAPPPAAAKYXRXXXRQXRX

SEQ ID NO: 319 - protein, 45 aa, Artificial
  Synthetic
YXRXXXRQXRXMEIPVPVQPSWLRRAXAPLPXXXYXRXXXRQXRX

SEQ ID NO: 320 - protein, 162 aa, Artificial
  Synthetic
XMEIPVPVQPSWLRRAXAPLPGLSAPGRLFDQRFGEGLLEAELAALCPTTLAPYYLRAPSVALPVAQVPTDPGHFSVLLDVKHFSPEEIAVKVVGEHVEVHARHEERPDEHGFVAREFHRRYRLPPGVDPAAVTSALSPEGVLSIQAAPASAQAPPPAAAKX

SEQ ID NO: 321 - protein, 24 aa, Artificial
  Synthetic
XMEIPVPVQPSWLRRAXAPLPXXX